(19) United States
(12) Patent Application Publication
(10) Pub. No.: US 2018/0311353 A1
(43) Pub. Date: Nov. 1, 2018

(54) TRIPLET-TRIPLET ANNIHILATION-BASED UPCONVERSION

(71) Applicant: Children's Medical Center Corporation, Boston, MA (US)

(72) Inventors: Daniel S. Kohane, Newton, MA (US); Weiping Wang, Jamaica Plain, MA (US); Qian Liu, Jamaica Plain, MA (US)

(73) Assignee: Children's Medical Center Corporation, Boston, MA (US)

(21) Appl. No.: 15/740,396

(22) PCT Filed: Jun. 30, 2016
(86) PCT No.: PCT/US2016/040271

§ 371 Date: Dec. 28, 2017

Related U.S. Application Data
(60) Provisional application No. 62/340,459, filed on May 23, 2016.
(60) Provisional application No. 62/188,077, filed on Jul. 2, 2015.

(51) Int. Cl. A61K 41/00; A61K 9/107; A61K 31/336; A61K 31/09; A61K 31/573; A61K 31/704; A61K 31/4745; A61K 31/409; A61K 9/00; A61K 47/69; A61P 35/00; A61N 5/06

(52) U.S. Cl. CPC A61K 41/0042; A61N 5/062; A61K 31/336; A61K 31/09; A61K 31/573; A61K 31/704; A61K 31/4745; A61K 31/409; A61K 9/0048; A61K 9/0014; A61K 9/0019; A61K 47/6937; A61K 47/6907; A61P 35/00; A61K 9/1075

(57) ABSTRACT

The present invention generally relates to various photoreactions, including reactions generally based on triplet-triplet annihilation upconversion. One aspect of the present invention is directed to systems and methods for absorbing energy (e.g., from a photon) in a photo sensitizer, transferring that energy by triplet-triplet energy transfer to an annihilator to produce a higher energy state via upconversion, then transferring that energy to cleave a cleavable or other active moiety, for instance, in order to cause the release of a releasable moiety. The energy may be transferred to the moiety via Forster resonance energy transfer. In some cases, these may be contained within a suitable carrier material, for example, a particle or a micelle. Such systems and methods may be used in a variety of applications, including various biological or physical applications. For example, such systems and methods may be useful for delivering drugs or other releasable moieties to regions of the body which may be affected by too much light, such as the eye. Other aspects of the present invention are generally directed to methods for making or using such systems, kits including such systems, or the like.

### RELATED APPLICATIONS

This application claims the benefit of U.S. Provisional Patent Application Ser. No. 62/188,077, filed Jul. 2, 2015, entitled “Triplet-Triplet Annihilation-Based Upconversion,” by Kohane, et al., and the benefit of U.S. Provisional Patent Application Ser. No. 62/340,459, filed May 23, 2016, entitled “Triplet-Triplet Annihilation-Based Upconversion,” by Kohane, et al. Each of these is incorporated herein by reference in its entirety for all purposes.

### GOVERNMENT FUNDING

This invention was made with government support under Grant No. GM073626 awarded by National Institutes of Health. The government has certain rights in the invention.

### FIELD

The present invention generally relates to various photoreactions, including reactions generally based on triplet-triplet annihilation upconversion.

### BACKGROUND

Spatiotemporal control over nanocarrier targeting and drug release would be desirable, for example, to enhance therapy while minimizing side effects. Light may be used to control the binding of nanocarriers to target cells (phototargeting). Conjugation of a photocleavable group (i.e. caging) may deactivate the binding activity of ligands on the nanocarrier surface. Irradiation at the appropriate wavelength removes the caging group and exposes the ligand, enabling binding to target cells. However, photocleavage generally requires short wavelength (high-energy) light, which can cause phototoxicity. Long-wavelength (low-energy) light that is less toxic cannot trigger photocleavage. Strategies for converting long-wavelength light into short-wavelength light are thus of interest, because they may allow photocleavage reactions with less phototoxicity.

### SUMMARY

The present invention generally relates to various photoreactions, including reactions generally based on triplet-triplet annihilation upconversion. The subject matter of the present invention involves, in some cases, interrelated products, alternative solutions to a particular problem, and/or a plurality of different uses of one or more systems and/or articles.

In one aspect, the present invention is generally directed to a composition. In one set of embodiments, the composition comprises a photosensitizer, an annihilator able to accept triplet-triplet energy transfer from the photosensitizer, a cleavable moiety able to accept energy from the annihilator in the higher energy state to cause cleavage of the cleavable moiety, and a releasable moiety releasable from the composition upon cleavage of the cleavable moiety.

In another set of embodiments, the composition comprises a carrier material comprising a photosensitizer, an annihilator, a cleavable moiety, and a releasable moiety. In some cases, absorption of an incident photon by the photosensitizer causes energy transfer to the annihilator and then to the cleavable moiety to cause cleavage of the cleavable moiety to release the releasable moiety from the carrier material, wherein the energy of the incident photon is insufficient to cause direct cleavage of the cleavable moiety.

The composition, in yet another set of embodiments, comprises a carrier material comprising a photosensitizer, an annihilator, an active moiety, and a releasable moiety. In some embodiments, absorption of an incident photon by the photosensitizer causes energy transfer to the annihilator and then to the active moiety to cause a chemical reaction within the active moiety, wherein the energy of the incident photon is insufficient to cause the chemical reaction in the active moiety.

In still another set of embodiments, the composition comprises a photosensitizer having an absorption, an annihilator able to receive energy from the photosensitizer to produce an upconversion emission having higher energy than the absorption of the photosensitizer, a cleavable moiety having an absorption overlapping with the upconversion emission from the annihilator, and a releasable moiety releasable from the composition upon cleavage of the cleavable moiety.

According to another set of embodiments, the composition comprises a photosensitizer having an absorption, an annihilator able to receive energy from the photosensitizer to produce an upconversion emission having higher energy than the absorption of the photosensitizer, and an active moiety having an absorption overlapping with the upconversion emission from the annihilator.

In one set of embodiments, the composition comprises a photosensitizer able to absorb a photon to produce higher energy state, an annihilator able to accept triplet-triplet energy transfer from the photosensitizer after absorption of the photon to produce a higher energy state via triplet-triplet annihilation, a cleavable moiety able to accept energy from the annihilator in the higher energy state to cause cleavage of the cleavable moiety, and a releasable moiety releasable from the composition upon cleavage of the cleavable moiety.

According to another set of embodiments, the composition comprises a photosensitizer able to absorb a photon to produce a higher energy state, an annihilator able to accept triplet-triplet energy transfer from the photosensitizer after absorption of the photon to produce a higher energy state via triplet-triplet annihilation, and a receiving moiety able to accept energy from the annihilator in the higher energy state via Förster resonance energy transfer.

The composition, in yet another set of embodiments, comprises a photosensitizer able to absorb a photon to produce a higher energy state, an annihilator able to accept triplet-triplet energy transfer from the photosensitizer after absorption of the photon to produce a higher energy state via triplet-triplet annihilation, a receiving moiety able to accept energy from the annihilator in the higher energy state via Förster resonance energy transfer, and a cleavable moiety able to accept energy from the annihilator in the higher energy state to cause cleavage of the cleavable moiety.

In still another set of embodiments, the composition comprises a photosensitizer, an annihilator able to accept triplet-triplet energy transfer from the first photosensitizer after absorption of a photon to produce a higher energy state via triplet-triplet annihilation, and a receiving moiety able to accept energy from the annihilator in the higher energy state.

The composition, in yet another set of embodiments, includes a photosensitizer able to absorb a photon, an annihilator able to accept triplet-triplet energy transfer from the photosensitizer, and a receiving moiety able to accept energy from the annihilator in the higher energy state.

The present invention, in another aspect, is generally drawn to a method. In accordance with one set of embodiments, the method includes absorbing a photon in a photosensitizer, transferring energy from the photosensitizer to an annihilator via triplet-triplet energy transfer, producing a higher-energy state via triplet-triplet annihilation from the transferred energy in two annihilators, transferring energy from the annihilator in the higher-energy state to an active moiety via Förster resonance energy transfer, and causing a chemical reaction in the active moiety using the transferred energy.

The method, in another set of embodiments, includes applying, to an eye of a subject, a composition comprising a photosensitizer, an annihilator able to accept triplet-triplet energy transfer from the photosensitizer, and a cleavable moiety able to accept energy from the annihilator in the higher energy state to cause cleavage of the cleavable moiety, and applying light to at least a portion of the eye to cause cleavage of the cleavable moiety.

In still another set of embodiments, the method includes applying, to an eye of a subject, a composition comprising a photosensitizer, an annihilator, a cleavable moiety, and a carrier material, and applying light to at least a portion of the eye. In some cases, absorption of light by the photosensitizer causes energy transfer to the annihilator and then to the cleavable moiety to cause cleavage of the cleavable moiety.

The method, in yet another set of embodiments, includes applying, to an eye of a subject, a composition comprising a carrier material comprising a photosensitizer having an absorption, an annihilator able to receive energy from the photosensitizer to produce an upconversion emission having higher energy than the absorption of the photosensitizer, and a cleavable moiety having an absorption overlapping with the upconversion emission from the annihilator, and applying light to at least a portion of the eye to cause cleavage of the cleavable moiety.

The method, in one set of embodiments, includes acts of absorbing an incident photon in a photosensitizer contained within a carrier material, transferring energy from the photosensitizer to an annihilator; and transferring energy from the annihilator to a cleavable moiety, wherein the average energy of the incident photon is insufficient to cause direct cleavage of the cleavable moiety.

In another set of embodiments, the method includes acts of absorbing a photon in a photosensitizer, transferring energy from the photosensitizer to an annihilator via triplet-triplet energy transfer, producing a higher-energy state via triplet-triplet annihilation from the transferred energy in two annihilators, and transferring energy from the annihilator in the higher-energy state to a receiving moiety.

The method, in accordance with yet another set of embodiments, includes applying, to a subject, a composition comprising a photosensitizer, an annihilator able to accept triplet-triplet energy transfer from the photosensitizer, and a cleavable moiety able to accept energy from the annihilator in the higher energy state to cause cleavage of the cleavable moiety, and applying light to at least a portion of the subject to cause cleavage of the cleavable moiety.

According to still another set of embodiments, the method includes applying, to a subject, a composition comprising a material comprising a photosensitizer, an annihilator, a cleavable moiety, and a carrier material, and applying light to at least a portion of the subject, wherein absorption of light by the photosensitizer causes energy transfer to the annihilator and then to the cleavable moiety to cause cleavage of the cleavable moiety.

The method, in yet another set of embodiments, includes applying, to the skin of a subject, a composition comprising a composition comprising a photosensitizer, an annihilator, a cleavable moiety, and a carrier material, and applying light to at least a portion of the skin, wherein absorption of light by the photosensitizer causes energy transfer to the annihilator and then to the cleavable moiety to cause cleavage of the cleavable moiety.

According to another set of embodiments, the method includes acts of applying, to the skin of a subject, a composition comprising a carrier material comprising a photosensitizer having an absorption, an annihilator able to receive energy from the photosensitizer to produce an upconversion emission having higher energy than the absorption of the photosensitizer, and a cleavable moiety having an absorption overlapping with the upconversion emission from the annihilator, and applying light to at least a portion of the skin to cause cleavage of the cleavable moiety.

The method, in yet another set of embodiments, includes applying, to the eye of a subject, a composition comprising a composition comprising a photosensitizer, an annihilator, a cleavable moiety, and a carrier material, and applying light to at least a portion of the skin, wherein absorption of light by the photosensitizer causes energy transfer to the annihilator and then to the cleavable moiety to cause cleavage of the cleavable moiety.

According to another set of embodiments, the method includes acts of applying, to the eye of a subject, a composition comprising a carrier material comprising a photosensitizer having an absorption, an annihilator able to receive energy from the photosensitizer to produce an upconversion emission having higher energy than the absorption of the photosensitizer, and a cleavable moiety having an absorption overlapping with the upconversion emission from the annihilator, and applying light to at least a portion of the skin to cause cleavage of the cleavable moiety.

In still another set of embodiments, the method includes applying, to a tumor in a subject, a composition comprising a composition comprising a photosensitizer, an annihilator, a cleavable moiety, and a carrier material, and applying light to at least a portion of the tumor, wherein absorption of light by the photosensitizer causes energy transfer to the annihilator and then to the cleavable moiety to cause cleavage of the cleavable moiety.

The method, in another set of embodiments, includes acts of applying, to a tumor in a subject, a composition comprising a carrier material comprising a photosensitizer having an absorption, an annihilator able to receive energy from the photosensitizer to produce an upconversion emission having higher energy than the absorption of the photosensitizer, and a cleavable moiety having an absorption overlapping with the upconversion emission from the annihilator, and applying light to at least a portion of the tumor to cause cleavage of the cleavable moiety.

In another aspect, the present invention encompasses methods of making one or more of the embodiments described herein, for example, compositions comprising photosensitizers and annihilators. In still another aspect, the present invention encompasses methods of using one or more of the embodiments described herein, for example, compositions comprising photo sensitizers and annihilators.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1C illustrate various TTA-UC and FRET processes, in accordance with some embodiments of the invention;

FIGS. 2A-2C illustrate cleavage of c[R]GDfK, in one embodiment of the invention;

FIGS. 3A-3E illustrate certain micelles containing c[R]GDfK, in certain embodiments of the invention;

FIGS. 4A-4B illustrate certain FRET processes in accordance with some embodiments of the invention;

FIGS. 5A-5B illustrate certain photocleavage reactions, in another set of embodiments of the invention;

FIGS. 6A-6B illustrate analyses of cell binding and uptake of micelles, in one set of embodiments of the invention;

FIG. 7 illustrates absorption of c[R]GDfK and TTA-UC emission spectrum of the mixture of PdOEP and DPA, in certain embodiments of the invention;

FIG. 8 illustrates a chemical reaction for producing a block copolymer, in another embodiment of the invention;

FIG. 9 illustrates production of DEACM-PLA-mPEG, in yet another embodiment of the invention;

FIG. 10 illustrates self-assembly of a micellar nanoparticle, in still another embodiment of the invention;

FIG. 11 illustrates formation of PLA-PEG-cRGDfK, in accordance with one embodiment of the invention;

FIGS. 12A-12B illustrate spectroscopy of NP_{TTA}, in another embodiment of the invention;

FIG. 13 illustrates synthesis of a conjugate, in still another embodiment of the invention;

FIG. 14 illustrates confocal laser scanning microscopy of micelle binding, in one embodiment of the invention;

FIG. 15 illustrates a synthesis reaction in accordance with another embodiment of the invention;

FIGS. 16A-16D illustrate in vivo use of a composition in still another embodiment of the invention;

FIGS. 17A-17B illustrate FRET and photocleavage of NP_{TTA}-c[R]GDfK, in one embodiment of the invention;

FIGS. 18A-18C illustrate in vitro phototargeting, in another embodiment of the invention;

FIGS. 19A-19C illustrate in vivo light triggering of nanoparticles, in yet another embodiment of the invention; and

FIGS. 20A-20C illustrate in vivo phototargeting in accordance with another embodiment of the invention.

### DETAILED DESCRIPTION

The present invention generally relates to various photoreactions, including reactions generally based on triplet-triplet annihilation upconversion. One aspect of the present invention is directed to systems and methods for absorbing energy (e.g., from a photon) in a photosensitizer, transferring that energy by triplet-triplet energy transfer to an annihilator to produce a higher energy state via upconversion, then transferring that energy to cleave a cleavable or other active moiety, for instance, in order to cause the release of a releasable moiety. The energy may be transferred to the moiety via Förster resonance energy transfer. In some cases, these may be contained within a suitable carrier material, for example, a particle or a micelle. Such systems and methods may be used in a variety of applications, including various biological or physical applications. For example, such systems and methods may be useful for delivering drugs or other releasable moieties to regions of the body which may be affected by too much light, such as the eye. Other aspects of the present invention are generally directed to methods for making or using such systems, kits including such systems, or the like.

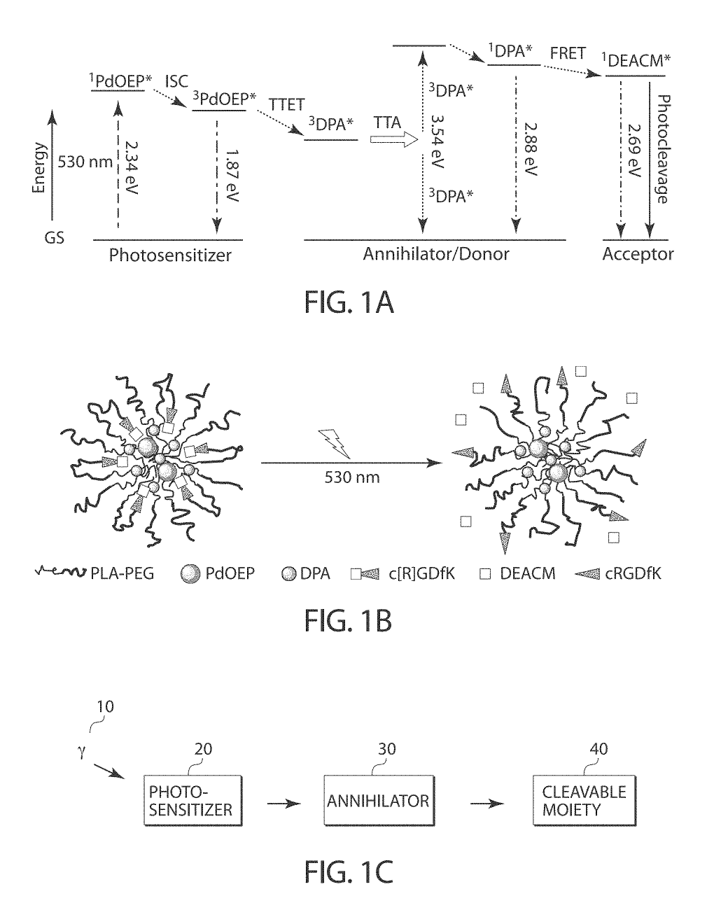

One aspect of the invention is now described with respect to FIG. 1C as a non-limiting example. A photon (γ) (e.g., 10 in FIG. 1C) may be absorbed by a photosensitizer (e.g., 20 in FIG. 1C), which may produce a higher-energy state in the photosensitizer. Under certain conditions, the photosensitizer may form a triplet state, either directly, or through intersystem crossing (ISC) from a singlet state produced through absorption of the photon.

It should be understood that, as is known to those of ordinary skill in the art, terms such as “singlet” or “triplet” generally refer to the electronic state of a molecule, not to the number of electrons that are present within the molecule. For example, in a singlet state, all of the electron spins within a molecule are typically paired such that the net spin the molecule has is 0, while in a triplet state, the molecule may have unpaired electrons present such that the net spin the molecule has is 1. Absorption of energy by a molecule, e.g., through absorption of a photon, may result in an electron from the molecule being “raised” from a lower energy state (or shell) to a higher energy state (or shell), which may alter the net spin of the molecule, while emission or transfer of that energy may allow a higher-energy electron to return to a lower state.

For example, in some cases, the energy from the triplet state of the photosensitizer may be transferred to an annihilator, as is shown as 30 in FIG. 1C. A variety of mechanisms may be involved in the transfer of such energy, such as triplet-triplet energy transfer (TTET). For instance, triplet-triplet energy transfer may be accomplished through the exchange of electrons that carry different spin and energy, e.g., between two molecules (such as between an annihilator and a photosensitizer), or between different parts of the same molecule. After such transfer, the annihilator may be in a higher-energy state, such as a triplet state, e.g., due to the presence of the exchanged electron.

In some cases, more than one annihilator molecule is in such a triplet state, e.g., via the absorption of several photons by one or more photosensitizers. In some embodiments, the energies from such annihilator molecules may be combined through quantum mechanical processes such as triplet-triplet annihilation. For instance, one of the annihilator molecules may end up in a low energy or in the ground state, while the other annihilator molecules may end up with substantially more energy, e.g., due to the combination of energy from the two annihilator molecules. In some embodiments, this energy may be greater than the energy of the original incident photon. Thus, for example, two photons of relatively low energy may by themselves have insufficient energy to produce a higher energy state, but through processes such as triplet-triplet-annihilation, may be combined to produce a higher energy state.

In addition, the energy from the higher energy state of the annihilator, after triplet-triplet annihilation, may be transmitted to another moiety, such as to a cleavable moiety (e.g., 40 in FIG. 1C). The cleavable moiety may then be cleaved as a result of the energy from the annihilator. The energy transfer from the annihilator to the cleavable moiety may occur through a variety of processes. For example, in one set of embodiments, energy transfer may occur via Förster resonance energy transfer (FRET). Surprisingly, FRET processes have not previously been suggested as a mechanism for transferring energy from an annihilator to a cleavable moiety. In FRET, energy transfer may occur between two molecules (which may be light-sensitive molecules or chromophores), through processes such as dipole-dipole coupling of the molecules. In some cases, transfer of energy may occur through emission (e.g., of a photon) by the annihilator and its absorption by the cleavable moiety; thus, for instance, the upconversion emission spectrum of the annihilator may overlap with the absorption spectrum of the cleavable moiety in order to facilitate such transfer.

Cleavage of the cleavable moiety can cause breakage of one or more bonds (e.g., covalent bonds) within or linked to the cleavable moiety. In some cases, cleavage of the cleavable moiety may cause a portion of the moiety to become separated or released, e.g., as a releasable moiety. Thus, in such a fashion, absorption of a photon (e.g., via a photosensitizer) may produce a chain of events that results in the release of releasable moiety. Accordingly, by controlling the incident light, the release of releasable moiety can be controlled as desired. However, it should be understood that a releasable moiety is not required, for example, cleavage of the cleavable moiety may result in other chemical or structural changes within the cleavable moiety. In addition, it should be understood that the energy may be transferred to other active moieties instead of a cleavable moiety, e.g., the energy may result in photoisomerization, rearrangement, photocycloaddition, or other chemical reactions.

Thus, in one set of embodiments, a composition comprising a photosensitizer, an annihilator, and a cleavable moiety (or other active moiety) may be applied to a region (e.g., within a sample, within a subject, etc.), and light applied to the region (or at least a portion of the region) in order to cause cleavage of the cleavable moiety, for example, to cause a chemical change, to release a releasable moiety, or the like. As mentioned, other active moieties may also be used. For example, if the active moiety is a cleavable moiety, the releasable moiety may be a drug, and light may applied to thereby cause release of the drug. As another non-limiting example, the releasable moiety can be a tracer (for example, a radioactive tracer, an inert molecule, a detectable entity, etc.) that can be introduced to a system (e.g., a biological system such as a cell or an organism, or a non-biological system such as a polymer), and the tracer released at an appropriate time (e.g., through applying light), for instance, instead of being instantly released upon administration or incorporation of the composition. The tracer may then be detected using any suitable technique, e.g., fluorescence, radioactivity, biological assay, chemical or enzymatic activity, etc.

In some cases, components such as the photosensitizer, the annihilator, and/or the cleavable moiety may be contained within a suitable carrier material. In some cases, the carrier material may hold the photosensitizer, the annihilator, and/or the cleavable moiety in close proximity to each other, e.g., to allow for electron and/or photon transfers to occur as discussed herein. For example, in one embodiment, the photosensitizer, the annihilator, and/or the cleavable moiety may be contained within a particle, such as a microparticle or a nanoparticle. In some cases, the particle may contain an environment (e.g., a hydrophobic or nonpolar environment), for instance, to keep the photosensitizer, the annihilator, or the cleavable moiety in close proximity, to facilitate transfer of electrons and/or photons, etc.

One specific non-limiting example of such a system is depicted in FIG. 1A. In this figure, PdOEP is palladium octaethylporphyrin, which may be excited by an incident photon to produce an excited singlet state, which may then produce an excited triplet state through intersystem crossing (ISC). The excited triplet state may exchange energy through triplet-triplet energy transfer with DPA or 9,10-diphenylanthracene. Two DPA molecules in the excited triplet state may participate in triplet-triplet annihilation (TTA) to produce one DPA molecule in the ground state and another in an even high energy state. For instance, as is shown here, the incident photon absorbed by PdOEP had an energy of 2.34 eV, but after these processes, one DPA molecule now has an energy of 3.54 eV, greater than the original 2.34 eV photon. In some cases, the excited triplet may then form an excited singlet state, for example, via intersystem crossing (ISC) or similar processes. The excited DPA molecule can then transfer energy to DEACM, or (7-diethylaminocoumarin-4-yl)-methyl) in this non-limiting example. The transferred energy may cause cleavage of DEACM in the process of returning to the ground state, i.e., the transferred energy causes the breakage or cleavage of a bond linked to DEACM. As shown in FIG. 1A, the transfer process may occur through Förster resonance energy transfer or FRET.

However, it should be understood that the above-described system of PdOEP, DPA, and DEACM is an illustrative example and is not limiting. In other aspects, a variety of other systems able to produce a higher energy state and cause cleavage of cleavable moieties (or other chemical reactions, e.g., with active moieties) via triplet-triplet annihilation upconversion and FRET processes are discussed in detail herein. These may include, for example, various photosensitizers, annihilators, cleavable moieties (which may include releasable moieties in certain embodiments) or other active moieties, carrier materials, or the like.

For instance, in one set of embodiments, the composition includes a photosensitizer. The photosensitizer can be any composition that is able to absorb a photon to produce a higher energy state. The higher energy state is a singlet excited state in some embodiments. The energy may be transferrable to the annihilator. In some cases, the photosensitizer is able to absorb a wavelength of visible light, i.e., about 390 to about 700 nm. However, in some instances, ultraviolet light (e.g., about 100 nm to about 400 nm) or infrared light (e.g., about 650 nm to about 1350 nm, or about 700 nm to about 1200 nm, etc.) may be absorbed by the photosensitizer.

As non-limiting examples, the photosensitizer may have an excitation wavelength of at least about 360 nm, at least about 370 nm, at least about 380 nm, at least about 390 nm, at least about 400 nm, at least about 410 nm, at least about 420 nm, at least about 430 nm, at least about 440 nm, at least about 450 nm, etc. In some embodiments, the photosensitizer has an excitation wavelength of no more than about 700 nm, no more than about 690 nm, no more than about 680 nm, no more than about 670 nm, no more than about 660 nm, no more than about 650 nm, no more than about 640 nm, no more than about 630 nm, no more than about 620 nm, no more than about 610 nm, no more than about 600 nm, etc. Combinations of any of these are also possible; for instance, the photosensitizer may have an excitation wavelength of between about 360 nm and about 700 nm, between 400 nm and about 700 nm, between 450 nm and about 700 nm, etc. It should be understood that the photosensitizer can be excited by light of a single wavelength (e.g., monochromatic light, such as would be supplied by a laser), or by light of different wavelengths (e.g., from a light source producing a spectrum of wavelengths).

In some embodiments, the photosensitizer is a compound that can be excited to the triplet excited state. In some cases, the photosensitizer is directly excited to an excited triplet state, although in other embodiments, the photosensitizer is first excited to an excited singlet state, and intersystem crossing or other suitable processes may convert the excited singlet state to an excited triplet state. Thus, the photosensitizer may exhibit, in some embodiments, absorption of the excitation light, a relatively high yield of intersystem crossing (ISC) for efficient production of the triplet state, and a relatively long triplet lifetime state (e.g., greater than microseconds). In addition, in some embodiments, the photosensitizer may have a large emax at the excitation wavelength in the visible-to-near-IR region of the spectrum. The triplet excited state of the photosensitizer can also be greater than the triplet acceptor energy of the annihilator in some cases.

The photosensitizer is a fluorophore in some embodiments. For example, in some cases, the photosensitizer includes a transition metal complex with a relatively large molar absorption coefficient in the visible spectral region. Non-limiting examples of transition metals useful in photosensitizers include Ir, Pd, Pt, Ru, Zn, Rh, Cu, or Au. A variety of triplet photosensitizers are known to those of ordinary skill in the art; many of these are commercially available. For example, the photosensitizer may be a phthalocyanine or a conjugated polymer. In one embodiment, the photosensitizer is porphyrin or a porphyrin derivative, e.g., a transition metal-porphyrin such as a Pt porphyrin or a Pd porphyrin. Specific non-limiting examples of photosensitizers include palladium octaethylporphyrin (PdOEP), platinum octaethylporphyrin (PtOEP), diiodoboron dipyrromethene (BODIPY-1), tris(2-phenylpyridinato-C²,N) iridium (III) (Ir(ppy)₃), platinum (II) tetraphenyltetrabenzoporphyrin (PtTPBP), 1,4,8,11,15,18,22,25-octabutoxyphthalocyaninato-palladium (II) (PdPc(OBu)8), 2,6-diiodoBodipy, etc. In another embodiment, the photosensitizer is BODIPY (4,4-difluoro-4-bora-3a,4a-diaza-s-indacene) or a BODIPY derivative, such as BODIPY FL, BODIPY R6G, BODIPY TMR, BODIPY581/591, BODIPY TR, BODIPY 630/650, BODIPY650/665, etc.

The photosensitizer may be in close proximity to an annihilator. For instance, the photosensitizer can be positioned such that energy may be transferred from the photosensitizer to the annihilator through a triplet-triplet energy transfer (TTET) process. In some cases, the photosensitizer can be directly covalently bound to an annihilator, or indirectly immobilized to an annihilator, e.g., through covalent binding to one or more linking entities between the photosensitizer and the annihilator. However, in other embodiments, the photosensitizer and the annihilator may not necessarily be immobilized using covalent bonds to each other, but are physically positioned within close proximity, e.g., such that electrons may be transferred between the photosensitizer and the annihilator. For example, both the photosensitizer and the annihilator may be contained within a carrier material, for example, contained within a liposome, a polymer film, a particle, a micelle, or the like.

The annihilator may be any composition that is able to accept triplet-triplet energy transfer from the photosensitizer. In some cases, the annihilator is able to upconvert the energy transferred from the photosensitizer via triplet-triplet annihilation. The annihilator may also be able to transfer the upconverted energy to the cleavable moiety, e.g., using FRET or other suitable processes. In some cases, the annihilator may have a fluorescent quantum yield of near 1. In some embodiments, the photosensitizer molecule is chosen so that its singlet excited state lies below that of the annihilator's singlet state while the photosensitizer's triplet state lies above that of the annihilator's. Thus, the singlet and triplet excited states of the photosensitizer can be nested between the singlet and triplet excited states of the annihilator, at least in some cases.

Typically, during upconversion, two molecules (e.g., two annihilator molecules), each in a triplet state, may react to produce two singlet states. This can generally be referred to as triplet-triplet annihilation (TTA). An interaction between the two molecules may be able to excite one of them to a higher energy singlet state, while the other molecule enters a lower energy singlet state. Essentially, the energy is combined together into one molecule to cause it to reach a higher, upconverted energy state, at the expense of the other molecule, which thereby returns to the ground state (or at least a lower energy state). Thus, triplet-triplet annihilation can be used to produce energy levels that are higher (“upconverted”) than the energy from the initial incident photons. This may be advantageously used, for example, in situations where higher energy states are desired, without using photons having too high of an energy level.

Thus, the annihilator may be a composition that is able to accept energy from a photosensitizer and upconvert that energy to produce a higher single excitation state. In some cases, the annihilator is a fluorophore. The fluorophore can include one or more fused benzene rings and/or one or more conjugated double bonds. The annihilator may also be able to emit that energy, e.g., as a photon, and/or through processes such as FRET. Non-limiting examples of annihilators include 9,10-diphenylanthracene (DPA), 3,8-di-tert-butylpyrene, perylene, 9,10-bis(diphenylphosphoryl)-anthracene, 4,4-difluoro-8-(4-iodophenyl)-1,3,5,7,-tetramethyl-4-bora-3a,4a-diaza-s-indacene (BODIPY-2), rubrene, tetraphenyl-pyrene (TPPy), or the like. In some cases, the annihilator is BODIPY (4,4-difluoro-4-bora-3a,4a-diaza-s-indacene) or a BODIPY derivative, e.g., an iodophenyl BODIPY derivative. The annihilator may be anthracene or an anthracene derivative in some cases. In some embodiments, the annihilator is a rubrene derivative.

In some embodiments, the annihilator has an emission wavelength of at least about 360 nm, at least about 370 nm, at least about 380 nm, at least about 390 nm, at least about 400 nm, at least about 410 nm, at least about 420 nm, at least about 430 nm, at least about 440 nm, at least about 450 nm, etc. In some embodiments, the photosensitizer has an excitation wavelength of no more than about 700 nm, no more than about 690 nm, no more than about 680 nm, no more than about 670 nm, no more than about 660 nm, no more than about 650 nm, no more than about 640 nm, no more than about 630 nm, no more than about 620 nm, no more than about 610 nm, no more than about 600 nm, etc. Combinations of any of these wavelengths are also possible in other embodiments; for instance, the photosensitizer may have an excitation wavelength of between about 360 nm and about 700 nm, between about 400 nm and about 510 nm, between about 410 nm and about 520 nm, between about 430 nm and about 600 nm, between about 500 nm and about 600 nm, between about 510 nm and about 700 nm, between about 360 nm and about 425 nm, etc. The annihilator may be able to emit photons at a single wavelength, or at more than one wavelength, depending on the annihilator. In some cases, the annihilator may be chosen such that the annihilator has an emission wavelength (or wavelengths) lower than the excitation wavelength of the photosensitizer.

Specific non-limiting examples of photosensitizer/annihilator pairs include Ir(ppy)₃/3,8-di-tert-butylpyrene, PdOEP/DPA, PtOEP/DPA, BODIPY-1/perylene, PtTPBP/9,10-bis(diphenylphosphoryl)-anthracene, PdTPBP/9,10-bis(diphenylphosphoryl)-anthracene, PtTPBP/BODIPY-2, PdTPBP/BODIPY-2, PdPc(OBu)₈/rubrene, PtPc(OBu)₈/rubrene, etc. Those of ordinary skill in the art will be aware of a variety of additional photosensitizer/annihilator or TTA (triplet-triplet annihilation) pairs. Other non-limiting examples include PtTPBP/perylene, (Ru(dmb)₃)²⁺/anthracene, BdTAP/rubrene, PtTPBP/2CBPEA (2-chloro-bis-phenylethynylanthracene), PdPH4TBP (palladium meso-tetraphenyltetrabenzoporphyrin)/B PEA (9,10-bis(phenylethynyl)anthracene), PdOEP/DPA, PdPc(OBu)₈/rubrene, PtOEP/DPA, ZnTPP (zinc (II) tetraphenylporphyrin)/perylene, ZnTPP/coumarin-343, (Ru(dmb)₃)²⁺/DMA (9,10-dimethylanthracene), PdTPBP/BPEA, PdPH₄OMe₈TNP/rubrene, PdPH₄OMe₈TNP/BPEN (5,12-bis(phenylethynyl)naphthacene), 2MeOTX (2-methoxythioxanthone)/PPO (2,5-diphenyloxazole), Ir(ppy)₃/pyrene, Ir(ppy)₃/t-butylpyrene, PtTPBP/BODIPY-1, (Ru(dmb)₃)²⁺/DPA, PtTPBP/BODIPY-2, or PdPH₄TBP/rubrene.

As mentioned, the annihilator may be able to transfer energy to another molecule, such as a cleavable moiety or other active moiety, using nonradiative transfer processes such as Förster resonance energy transfer (FRET). Surprisingly, FRET has not previously suggested for transferring energy from one molecule (such as an annihilator) to another molecule (such as an active moiety). Typically in FRET processes, a first molecule, initially in an excited state (e.g., a “donor”), may transfer energy to an acceptor through nonradiative dipole-dipole coupling, which may form part of the cleavable moiety. The efficiency of this energy transfer is usually inversely proportional to the sixth power of the distance between the donor and the acceptor. The energy received by the active moiety can result in cleavage of one or more bonds within or linked to the acceptor, i.e., the acceptor can act as a cleavable moiety, or produce other chemical changes, e.g., photoisomerization, rearrangement, photocycloaddition, or other chemical reactions.

In some embodiments, the acceptor is fluorescent. For example, the acceptor moiety and the annihilator may each be fluorescent entities that are able to interact via FRET. Non-limiting examples of acceptors include arylcarbonylmethyl groups, 2-nitrobenzyl groups, or coumarin-4-ylmethyl groups. Specific non-limiting examples of coumarin-4-ylmethyl groups include HCM, (7-hydroxycoumarin-4-yl)methyl; MCM, (7-methoxycoumarin-4-yl)methyl; ACM, (7-acetoxycoumarin-4-yl)methyl; PCM, (7-propionyloxycoumarin-4-yl)methyl; DMCM, (6,7-dimethoxycoumarin-4-yl)methyl; BECMEM, (6,7-bis(ethoxycarbonylmethoxy)coumarin-4-yl)methyl; Bhc, (6-bromo-7-hydroxycoumarin-4-yl)methyl; DEACM, (7-diethylaminocoumarin-4-yl)methyl; DMACM, (7-dimethylaminocoumarin-4-yl)methyl, DEAC450, or thiocumarin. In one embodiment, the acceptor is a coumarin derivative. The acceptor may also be a DEACM derivative.

In certain embodiments, the acceptor has absorption that partially or completely overlaps with the upconversion emission from the annihilator. For example, the acceptor may have an absorption spectrum that includes wavelengths of at least about 360 nm, at least about 370 nm, at least about 380 nm, at least about 390 nm, at least about 400 nm, at least about 410 nm, at least about 420 nm, at least about 430 nm, at least about 440 nm, at least about 450 nm, etc. In some embodiments, the acceptor can have an absorption spectrum of no more than about 700 nm, no more than about 690 nm, no more than about 680 nm, no more than about 670 nm, no more than about 660 nm, no more than about 650 nm, no more than about 640 nm, no more than about 630 nm, no more than about 620 nm, no more than about 610 nm, no more than about 600 nm, etc. Combinations of any of these wavelengths are also possible; as non-limiting examples, the absorption may be between about 360 nm and about 700 nm, between about 400 nm and about 510 nm, between about 410 nm and about 520 nm, between about 430 nm and about 600 nm, between about 500 nm and about 600 nm, between about 510 nm and about 700 nm, between about 360 nm and about 425 nm, etc.

Without wishing to be bound by any theory, it is believed that the annihilator may be in an excited state and, when it relaxes to reach the lowest excited singlet state, energy released when the electron returns to the ground state may be non-radiatively transferred via resonance to the acceptor (for example, as part of a cleavable entity). This may be facilitated, for example, due to the overlapping spectra. The energy may then be directed to an active moiety, for instance, used to cleave a bond within or linked to a cleavable moiety, e.g., directly, or through production of a photon which then causes cleavage of a bond, or other chemical reactions such as are described herein.

Thus, in some embodiments, the acceptor (which may be contained within an active moiety such as a cleavable moiety) may be positioned such that FRET may be used to transfer energy nonradiatively between the annihilator and the acceptor. For example, the annihilator and the acceptor may be directly covalently bound to each other, or indirectly immobilized to each other, e.g., through covalent binding to one or more linking entities between the annihilator and the active moiety. However, in some cases, the annihilator and the acceptor may be physically positioned within close proximity to each other. For instance, the annihilator and the acceptor can be contained within a carrier material, for example, contained within a liposome, a polymer film, a particle, a micelle, or the like. In some cases, the annihilator and the acceptor can be positioned such that they are separated by a distance of less than about 15 nm, less than about 13 nm, less than about 12 nm, less than about 11 nm, less than about 10 nm, less than about 9 nm, less than about 8 nm, less than about 7 nm, less than about 6 nm, less than about 5 nm, less than about 4 nm, less than about 3 nm, less than about 2 nm, or less than about 1 nm from each other.

In some cases, the transfer of energy to the acceptor results in the cleavage of a bond within or linked to the acceptor, and/or within or linked to a different portion of a cleavable moiety containing the acceptor. Cleavage of the bond, in some embodiments, can cause the release of a portion of the cleavable moiety, e.g., as a releasable moiety. However, it should be understood that in other embodiments, the cleavage of a single bond does not necessarily require the release of a releasable moiety, for instance, if more than one bond connects portions of the molecule together. In addition, in some embodiments, transfer of energy to acceptor may result in other chemical reactions within the acceptor, not necessarily leading to the cleavage of a cleavable bond.

If present, a releasable moiety may be any suitable moiety that can be released, e.g., during cleavage (including photocleavage). The releasable moiety can include the acceptor, and/or a portion of the cleavable entity that is separate from the acceptor, but is cleaved as a result of the transfer of energy to the acceptor, e.g., via FRET. Different releasable moieties can be used in various embodiments, depending on the application. For example, the releasable moiety may include a drug, a tracer (e.g., a fluorescent or radioactive compound), a caged species, a peptide or protein, a small molecule (e.g., having a molecular weight of less than about 1 kDa or about 2 kDa), or the like. In some cases, the exact form of the releasable moiety is not critical, e.g., if it is attached through a cleavable bond of a cleavable moiety that itself is cleaved as discussed above; cleavage of the cleavable bond may thereby cause separation of the releasable moiety, regardless of the exact composition of the releasable moiety.

As non-limiting examples, in one set of embodiments, the releasable moiety can include an anti-angiogenesis drug, such as TNP-470 or Combretastatin A4. In another set of embodiments, the releasable moiety may include an anti-inflammatory drug, such as dexamethasone. In yet another set of embodiments, the releasable moiety includes an anticancer drug and/or a chemotherapy drug, such as doxorubicin, topotecan, or verteporfin. In yet another set of embodiments, the releasable moiety may include fluorescent proteins, such as GFP or YFP. In still another set of embodiments, the releasable moiety can include fluorescent compounds, such as fluorescein, rhodamine, or calcein. In still another set of embodiments, the releasable moiety includes a peptide or a protein, such as an RGD peptide. In another set of embodiments, the releasable moiety may include a radioactive atom, such as ³H, ¹¹C, ¹³N, ¹⁴C, ¹⁵O, ¹⁸F, ²⁴Na, ³²P, ³³P, ³⁵S, ³⁶Cl, ⁴⁶Sc, ⁵⁶Mn, ⁶⁰Co, ⁸⁹Sr, ⁹⁰Y, ^{99m}Tc, ¹⁰³Pd, ¹⁰⁶Ru, ¹²³I, ¹²⁵I, ¹²⁹I, ¹³¹I, ¹³⁷I, ¹³⁷O, ¹⁵³Sm, ¹⁷⁷Lu, or ¹⁹²Ir.

However, in other embodiments, the transfer of energy to the acceptor results in other changes within an active moiety. For instance, the transfer of energy may result in photoisomerization (e.g., of azobenzene-based, azotolane-based, spiropyran-based, or fulvalene diruthenium (FvRu₂) molecules), photo-induced Wolff rearrangement (e.g., of 2-diazo-1,2-naphthoquinone (DNQ) groups), or photocycloaddition (e.g., of [2+2] photocycloaddition of coumarin groups, e.g., coumarin groups such as those discussed herein). In another embodiment, the transfer of energy may be used to produce OH radical groups (.OH) or water splitting, e.g., using DPA/PdOEP systems.

In some embodiments, the photosensitizer, the annihilator, the active moiety (e.g., a cleavable moiety), and/or the releasable moiety (if present) are contained within a suitable carrier material. The carrier material may hold some or all of these in close proximity to each other (e.g., as discussed above). In some cases, the carrier material may create an environment favorable for compounds such as those discussed herein to be fluorescent and/or to be able to absorb electrons, photons, etc. as described herein. For example, the carrier material may create an aqueous environment, a hydrophobic environment, a polar or nonpolar environment, etc. In some cases, the carrier material creates an environment that repels water.

In one set of embodiments, the carrier material is formed from a polymer. Any suitable polymer can be used. Examples of polymers include, but are not limited to, polylactic acid, polyglycolic acid, polyethylene oxide, polystyrene, polyethylene, polypropylene, etc. In some embodiments, the polymer may be biodegradable or biocompatible, e.g., for use in various medical or biological applications. In some cases, more than one polymer can be used, and the polymers may be physically blended together and/or chemically combined, e.g., as in a copolymer. As a non-limiting example, the carrier material may include a copolymer such as poly(D,L-lactic acid)-poly(ethylene oxide). However, it should be understood that the carrier material need not be limited to polymeric materials. For example, in other embodiments, the carrier material can include silica, ceramics, or other materials.

The carrier material can be present in any suitable form. For example, the carrier material can be present as a film, as a block of material, as particles, as a micelle, or the like. In some cases, components such as the photosensitizer, the annihilator, the active moiety, and/or the releasable moiety may be added to the carrier material during and/or after formation of the carrier material. The carrier material can be formed using any suitable techniques; for example, techniques for producing polymers, silica gels, ceramics, etc. are known to those of ordinary skill in the art.

If the carrier material is present as particles, the particles may be spherical or nonspherical, and may have any suitable diameter. For instance, the particles may have an average diameter of less than about 1 mm, less than about 500 micrometers, less than about 300 micrometers, less than about 100 micrometers, less than about 50 micrometers, less than about 30 micrometers, less than about 10 micrometers, less than about 5 micrometers, less than about 3 micrometers, less than about 1 micrometer, less than about 500 nm, less than about 300 nm, less than about 100 nm, less than about 50 nm, less than about 30 nm, less than about 10 nm, etc. The average diameter of a nonspherical particle may be taken as the volume of a perfect sphere having the same volume of the particle.

If the carrier material is present as a film, the film can have any cross-sectional thickness. For example, the film may have an average thickness of less than about 1 mm, less than about 500 micrometers, less than about 300 micrometers, less than about 100 micrometers, less than about 50 micrometers, less than about 30 micrometers, less than about 10 micrometers, less than about 5 micrometers, less than about 3 micrometers, less than about 1 micrometer, less than about 500 nm, less than about 300 nm, less than about 100 nm, less than about 50 nm, less than about 30 nm, less than about 10 nm, etc.

The carrier material may also comprise one or more polymeric micelles. The polymer micelles may have any suitable average diameter. For example, the micelles can have an average diameter of less than about 1 mm, less than about 500 micrometers, less than about 300 micrometers, less than about 100 micrometers, less than about 50 micrometers, less than about 30 micrometers, less than about 10 micrometers, less than about 5 micrometers, less than about 3 micrometers, less than about 1 micrometer, less than about 500 nm, less than about 300 nm, less than about 100 nm, less than about 50 nm, less than about 30 nm, less than about 10 nm, etc.

As mentioned, compositions such as those discussed herein may be used in a wide variety of applications, including biological and medical applications, as well as non-biological or non-medical applications. As a non-limiting example, in one set of embodiments, a composition as discussed herein may be applied to a subject. The subject may be human or non-human. For example, the subject may be a rat, mouse, rabbit, goat, cat, dog, or the like. The composition can also be applied to any suitable sample, e.g., a biological sample, a physical sample, a chemical sample, or the like.

Light may be applied to the composition to cause release of the releasable moiety, if present. The light may be monochromatic light (e.g., laser or coherent light), or the light may be nonmonochromatic or noncoherent in some embodiments. The light may have any suitable frequency, e.g., including the frequencies discussed herein. In some cases, the light has a frequency such that the average energy of the incident light is insufficient to cause direct cleavage of the cleavable moiety or interact with an active moiety, but due to upconversion, etc., as discussed herein, the incident light may cause cleavage of the cleavable moiety, photoreaction within the active moiety, or release of the releasable moiety, etc.

In one set of embodiments, the light is applied at an irradiance of at least about 1 mW/cm², at least about 2 mW/cm², at least about 5 mW/cm², at least about 10 mW/cm², at least about 20 mW/cm², at least about 30 mW/cm², at least about 40 mW/cm², at least about 50 mW/cm², at least about 60 mW/cm², at least about 70 mW/cm², at least about 80 mW/cm², at least about 90 mW/cm², at least about 100 mW/cm², at least about 110 mW/cm², at least about 125 mW/cm², at least about 150 mW/cm², at least about 200 mW/cm², at least about 250 mW/cm², at least about 300 mW/cm², at least about 400 mW/cm², at least about 500 mW/cm², etc. In some cases, the light is applied at an irradiance of no more than about 1000 mW/cm², no more than about 500 mW/cm², no more than about 400 mW/cm², no more than about 300 mW/cm², no more than about 250 mW/cm², no more than about 200 mW/cm², no more than about 150 mW/cm², no more than about 125 mW/cm², no more than about 110 mW/cm², no more than about 100 mW/cm², no more than about 90 mW/cm², no more than about 80 mW/cm², no more than about 70 mW/cm², no more than about 60 mW/cm², no more than about 50 mW/cm², no more than about 40 mW/cm², no more than about 30 mW/cm², no more than about 20 mW/cm², no more than about 10 mW/cm², no more than about 5 mW/cm², no more than about 2 mW/cm², etc. Combinations of any of the above are also possible in certain embodiments. For instance, the light may be applied at an irradiance of between about 50 mW/cm² and about 150 mW/cm².

In one set of embodiments, the composition is applied to a subject to treat a tumor. The composition may be applied directly to the tumor, and/or applied systemically to the body of the subject such that at least some of the composition is able to travel to the tumor (e.g., via the blood) such that light can be applied to the tumor (or portion thereof), e.g., to cause release of a releasable moiety for determining and/or treating the tumor. The composition can include, for example, an anti-angiogenesis drug, an anti-inflammatory drug, a radioactive species, an anticancer drug and/or a chemotherapy drug, and light may be applied to the tumor to cause release. Such application may be targeted, e.g., by applying light directly to the tumor (or at least a portion thereof); thus, release elsewhere within the subject may be minimized by not applying light to other places. In such a fashion, release of a drug (or other suitable release moiety) may be controlled or localized at or near the tumor by applying light directly to the tumor (or portion thereof), or at least proximate the tumor. In some cases, more than one composition may be present.

In another set of embodiments, the composition may be applied to a subject for treatment to the eye. The eye is sensitive to light, and in fact, too much light may be harmful to the eye. Thus, by using compositions such as those described herein, in some cases, light intensities or irradiation to the eye can be minimized while still being able to cause cleavage of a cleavable moiety, reaction within an active moiety, and/or release of a releasable moiety. The subject may, for example, have various eye conditions in need of treatment, such as macular degeneration (e.g., age-related macular degeneration) or retinoblastoma. The composition can be applied directly to the eye, and/or applied systemically to the body such that at least some of the composition is able to travel to the eye (e.g., via the blood) such that light can be applied to the eye (or a portion of the eye) to interact with the composition as discussed herein. One or both eyes may be treated, depending on the condition of the subject.

Other portions of a subject may also be treated in various embodiments. For instance, the composition may be applied directly to a specific location within the subject, or applied systemically to the subject such that at least some of the composition is able to travel to a location where light is to be applied. For instance, the composition may be applied to the skin (or to the blood) and light applied to a portion of the skin to cause local release of a releasable moiety.

In various aspects, the compositions described herein can be administered by any suitable method, e.g., contained in a solution or suspension, such as inhalation solutions, local instillations, eye drops, intranasal introductions, an ointment for epicutaneous applications, intravenous solutions, injection solutions (e.g., subcutaneous, or intravenous), or suppositories. In one set of embodiments, the composition is introduced parenterally or topically. For instance, the composition may be contained within a cream, gel, or ointment applied to the skin. In some embodiments, the composition can be applied one or more times a day, by one or more administrations per day, by fewer than one time per day, or by continuous administration, etc., until a desired therapeutic effect is achieved.

In some embodiments, the composition is introduced to the subject at a dose from, e.g., 0.01 to 100.0 mg of the composition per kg of body weight of the subject. In some cases, the dose may be at least about 0.01 mg/kg, at least about 0.03 mg/kg, at least about 0.05 mg/kg, at least about 0.1 mg/kg, at least about 0.3 mg/kg, at least about 0.5 mg/kg, at least about 1 mg/kg, at least about 3 mg/kg, at least about 5 mg/kg, at least about 10 mg/kg, at least about 30 mg/kg, at least about 50 mg/kg, and/or no more than about 100 mg/kg, no more than about 50 mg/kg, no more than about 30 mg/kg, no more than about 10 mg/kg, no more than about 5 mg/kg, no more than about 3 mg/kg, no more than about 1 mg/kg, no more than about 0.5 mg/kg, no more than about 0.3 mg/kg, no more than about 0.1 mg/kg, no more than about 0.05 mg/kg, no more than about 0.03 mg/kg, etc. Where the composition is administered as a solution, the solution may have, for example, a concentration of between about 1% to about 10% of the composition. In one set of embodiments, the composition may be, or include, a pharmaceutically acceptable derivative, e.g., for parenteral use is in a pharmaceutically acceptable solvent such as, for example, an aqueous solution including water, glucose solution, isotonic solutions of sodium chloride, buffered salt solutions, or the like. Other physiological solvents or carriers can be used in other embodiments.

As mentioned, certain aspects of the present invention provide methods of administering any composition of the present invention to a subject. When administered, the compositions of the invention are applied in a therapeutically effective, pharmaceutically acceptable amount as a pharmaceutically acceptable formulation. As used herein, the term “pharmaceutically acceptable” is given its ordinary meaning. Pharmaceutically acceptable compositions are generally compatible with other materials of the formulation and are not generally deleterious to the subject. Any of the compositions of the present invention may be administered to the subject in a therapeutically effective dose. A “therapeutically effective” amount as used herein means that amount necessary to delay the onset of, inhibit the progression of, halt altogether the onset or progression of, diagnose a particular condition being treated, or otherwise achieve a medically desirable result. When administered to a subject, effective amounts will depend on the particular condition being treated and the desired outcome. A therapeutically effective dose may be determined by those of ordinary skill in the art, for instance, employing factors such as those further described below and using no more than routine experimentation.

Any medically acceptable method may be used to administer the composition to the subject. The administration may be localized (i.e., to a particular region, physiological system, tissue, organ, or cell type) or systemic, depending on the condition to be treated. For example, the composition may be administered orally, vaginally, rectally, buccally, pulmonary, topically, nasally, transdermally, through parenteral injection or implantation, via surgical administration, or any other method of administration. Examples of parenteral modalities that can be used with the invention include intravenous, intradermal, subcutaneous, intracavity, intramuscular, intraperitoneal, epidural, or intrathecal. Examples of implantation modalities include any implantable or injectable drug delivery system. Use of an implant may be particularly suitable in some embodiments of the invention. The implant containing the composition may be constructed and arranged to remain within the body for at least 30 or 45 days, and preferably at least 60 or 90 days, or even longer in some cases. Long-term release implants are well known to those of ordinary skill in the art.

In certain embodiments of the invention, a composition can be combined with a suitable pharmaceutically acceptable carrier, for example, as incorporated into a liposome, incorporated into a polymer release system, or suspended in a liquid, e.g., in a dissolved form, or a colloidal form, or a micellular form. In general, pharmaceutically acceptable carriers suitable for use in the invention are well-known to those of ordinary skill in the art. A pharmaceutically acceptable carrier may include non-toxic material that does not significantly interfere with the effectiveness of the biological activity of the active compound(s) to be administered, but is used as a formulation ingredient, for example, to stabilize or protect the active compound(s) within the composition before use. The carrier may be organic or inorganic, and may be natural or synthetic, with which one or more active compounds of the invention are combined to facilitate the application of the composition. The carrier may be either soluble or insoluble, depending on the application. Examples of well-known carriers include glass, polystyrene, polypropylene, polyethylene, dextran, nylon, amylase, natural and modified cellulose, polyacrylamide, agarose and magnetite. The nature of the carrier can be either soluble or insoluble. Those skilled in the art will know of other suitable carriers, or will be able to ascertain such, using only routine experimentation.

In some embodiments, the compositions of the invention include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers that may be used with the active compound. For example, if the formulation is a liquid, the carrier may be a solvent, partial solvent, or non-solvent, and may be aqueous or organically based. Examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. Suitable carrier concentrations can be determined by those of ordinary skill in the art, using no more than routine experimentation. The compositions of the invention may be formulated into preparations in solid, semi-solid, liquid or gaseous forms such as tablets, capsules, elixirs, powders, granules, ointments, creams, gels, pastes, solutions, depositories, inhalants, injectables, or the like. Those of ordinary skill in the art will know of other suitable formulation ingredients, or will be able to ascertain such, using only routine experimentation.

In another aspect, the present invention is directed to a kit including one or more of the compositions discussed herein. A “kit,” as used herein, typically defines a package or an assembly including one or more of the compositions of the invention, and/or other compositions associated with the invention, for example, as described herein. Each of the compositions of the kit may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components associated with the invention include, but are not limited to, solvents, surfactants, diluents, salts, buffers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, and the like, for example, for using, administering, modifying, assembling, storing, packaging, preparing, mixing, diluting, and/or preserving the compositions components for a particular use, for example, to a sample and/or a subject.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the composition and/or other compositions associated with the kit. In some cases, the instructions may also include instructions for the delivery and/or administration of the compositions, for example, for a particular use, e.g., to a sample and/or a subject. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

U.S. Provisional Patent Application Ser. No. 62/188,077, filed Jul. 2, 2015, entitled “Triplet-Triplet Annihilation-Based Upconversion,” by Kohane, et al., and U.S. Provisional Patent Application Ser. No. 62/340,459, filed May 23, 2016, entitled “Triplet-Triplet Annihilation-Based Upconversion,” by Kohane, et al. are each incorporated herein by reference in its entirety for all purposes.

The following examples are intended to illustrate certain embodiments of the present invention, but do not exemplify the full scope of the invention.

### Example 1

High-efficiency upconverted light would be a desirable stimulus for triggered drug delivery. This example presents a general strategy to achieve photoreactions based on triplet-triplet annihilation upconversion (TTA-UC) and Förster resonance energy transfer (FRET). This example designed poly(D,L-lactic acid)-poly(ethylene oxide) (PLA-PEG) micellar nanoparticles containing in their cores hydrophobic photosensitizer and annihilator molecules which, when stimulated with green light, would undergo TTA-UC. The upconverted energy was then transferred by FRET to a hydrophobic photocleavable group (DEACM), also in the core. The DEACM was bonded to (and thus inactivated) the cell-binding peptide cyclo-(RGDfK), which was bound to the PLA-PEG chain. Cleavage of DEACM by FRET re-activated the PLA-PEG-bound peptide and allowed it to move from the particle core to the surface. TTA-UC followed by FRET allowed photo-controlled binding of cell adhesion with green light LED irradiation at low irradiance for short periods. These are attractive properties in photo-triggered systems.

Triplet-triplet annihilation (TTA) is an upconversion process which can be driven by low-power noncoherent light sources (˜mW cm⁻²), which enhances safety and is of practical and economic benefit. In TTA-upconversion (TTA-UC; FIG. 1A), a low-energy photon is absorbed by a photosensitizer, which then undergoes intersystem crossing (ISC) to form a more stable triplet state. The triplet state energy of the photosensitizer is subsquently transferred to a molecule which is thus excited to its triplet state. Two such molecules in the triplet state can then combine their energies through TTA to form one molecule in the singlet state (with higher energy), and another in the ground state. (Those molecules are often termed annihilators because their interaction “annihilates” the triplet state.) The molecule in the singlet state can relax to the ground state, usually by emission of a higher-energy photon.

In this example, relaxation to the ground state can also be achieved by Förster resonance energy transfer (FRET). TTA-UC is coupled with FRET to create an upconversion-based photoresponsive nanoparticulate system. Incident long-wavelength light is efficiently upconverted to high energy through TTA, and transferred by FRET to a photocleavable group, triggering its cleavage; some of the energy may also go to emitting one or more photons. Cleavage of that bond removes the photocleavable (caging) group from a targeting ligand, restoring its binding activity. See FIG. 1.

This example uses palladium octaethylporphyrin (PdOEP, excitation=532 nm) as a photosensitizer and 9,10-diphenylanthracene (DPA, emission=400-500 nm) as the annihilator, due to their high upconversion efficiency. PdOEP and DPA were encapsulated in the hydrophobic core of a polymeric micelle self-assembled from the block copolymer poly(D,L-lactic acid)-poly(ethylene oxide) (PLA-PEG). The peptide cyclo-(RGDfK) (cRGDfK) was conjugated on the PEG end as the targeting group. cRGDfK was chosen because it binds preferentially to αᵥβ₃ (alpha-V-beta-3) integrin, which is overexpressed on tumor cells and angiogenic endothelial cells during tumor growth. Photocaging of this peptide with a 2-nitrobenzyl-based group has been used to regulate cell adhesion with UV light on solid surfaces in vitro and hydrogels in vivo.

A coumarin-based group, (7-diethylaminocoumarin-4-yl)-methyl (DEACM), was selected as the caging or photocleavable group, because of its high photocleavage efficiency and relatively long absorption wavelength (up to 455 nm, which overlaps with the emission spectrum of DPA, enabling FRET). It is believed that the hydrophobicity of the DEACM would place the DEACM-caged cRGDfK in the PLA core of the PLA-PEG micellar nanoparticles. Since FRET efficiency also depended on the distance between the donor and the acceptor, typically in the range of 1-10 nm, this arrangement (PdOEP, DPA and DEACM being in the core) would allow TTA-UC energy to be efficiently transferred to the DEACM through FRET (FIG. 1B), causing the removal of the hydrophobic caging group. Uncaging would allow the hydrophilic peptide to return to the micelle surface, allowing the binding of micelles to target cells.

FIG. 1 shows a schematic illustration of TTA-UC and FRET processes. FIG. 1A is a Jablonski diagram illustrating the mechanism of TTA-UC and FRET processes discussed above. GS: the ground state. ISC: intersystem crossing. TTET: triplet-triplet energy transfer. TTA: triplet-triplet annihilation. FRET: Förster resonance energy transfer. FIG. 1B is a schematic of the photo-triggering of the polymeric micellar nanoparticle by TTA-UC and FRET.

### Example 2

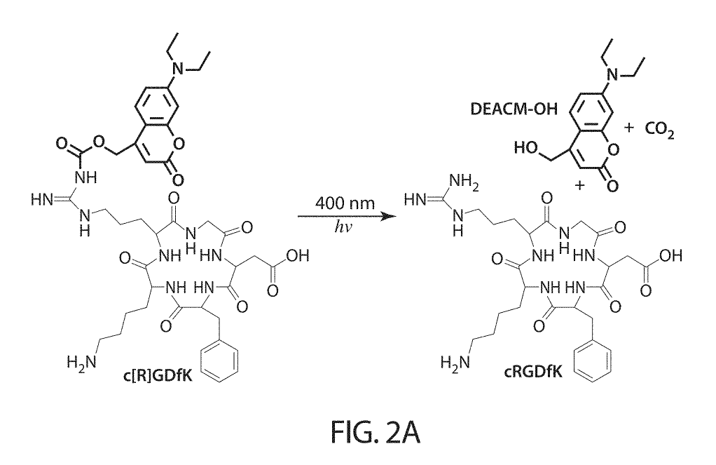

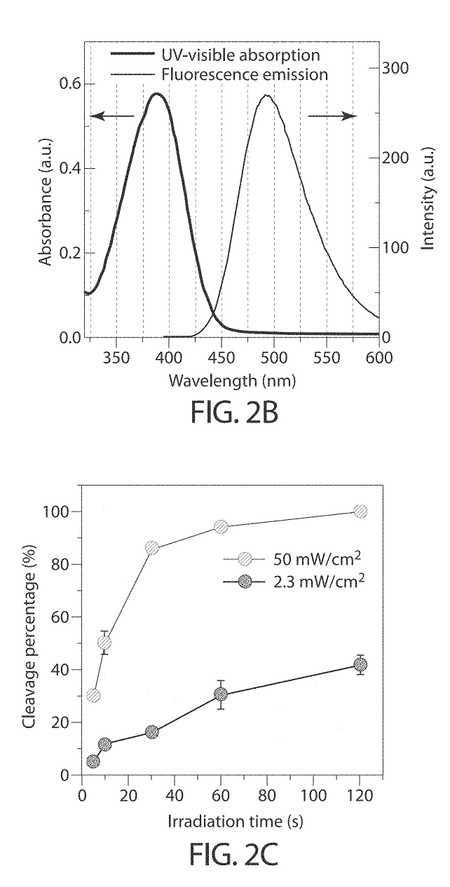

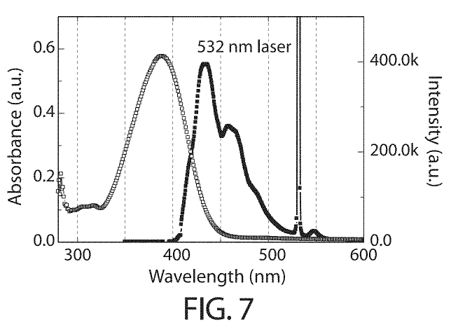

Construction of photo-targeted polymeric micelles. The photo-responsive cell-targeting portion of the micellar nanoparticle, DEACM-caged cRGDfK (c[R]GDfK; FIG. 2A), was synthesized and characterized in this example. c[R]GDfK showed a broad UV-visible absorption spectrum with a peak at 388 nm and a full width at half maximum (FWHM) of 31 nm (FIG. 2B) which overlapped with the TTA-UC emission spectrum from DPA (annihilator/donor; FIG. 7), suggesting the possibility of FRET between DPA (donor) and DEACM (acceptor). The DEACM group on the peptide had a fluorescence emission peak at 493 nm (FIG. 2B) when excited at 385 nm; this was used below to probe the polarity of the environment surrounding DEACM. Irradiation of c[R]GDfK with a 400 nm light-emitting diode (LED) at 50 mW cm⁻² for 1 min resulted in cleavage of c[R]GDfK: on high-performance liquid chromatography (HPLC), the c[R]GDfK peak decreased and two new peaks appeared that had the same elution times as free cRGDfK and 7-diethylamino-4-hydroxymethylcoumarin (DEACM-OH). The identity of the peaks was further confirmed by mass spectrometry. Approximately 86% of cleavage occurred within 30 s of irradiation at 50 mW cm⁻² (FIG. 2C). At an irradiance as low as 2.3 mW cm⁻², ˜42% of c[R]GDfK was cleaved after 2 min of irradiation. These results demonstrate that the photocleavage reaction could generate intact cRGDfK peptide after short periods of relatively low irradiances.

FIG. 2 shows photocleavage of c[R]GDfK. FIG. 2A. Photocleavage of c[R]GDfK. DEACM-OH and intact cRGDfK peptide were released upon irradiation at 400 nm. FIG. 2B. UV-visible absorption and fluorescence emission spectra of c[R]GDfK in PBS (pH 7.4). The excitation wavelength for the fluorescence spectrum was 385 nm. FIG. 2C. Photocleavage rate of c[R]GDfK in PBS, as determined by HPLC (detected at 390 nm), after continuous irradiation with 400 nm LED light at 2.3 mW cm⁻² and 50 mW cm⁻² (data are mean+/−SD; n=4). a.u.=arbitrary units. The concentration of c[R]GDfK in all samples was 50 microgram/mL. FIG. 7 shows the absorption spectrum of c[R]GDfK (50 μg mL⁻¹) in PBS (same trace as in FIG. 2B) and the TTA-UC emission spectrum of the mixture of sensitizer (PdOEP, 10 micromolar) and annihilator (DPA, 1.0 mM) in toluene. Excitation wavelength λ=532 nm.

### Example 3

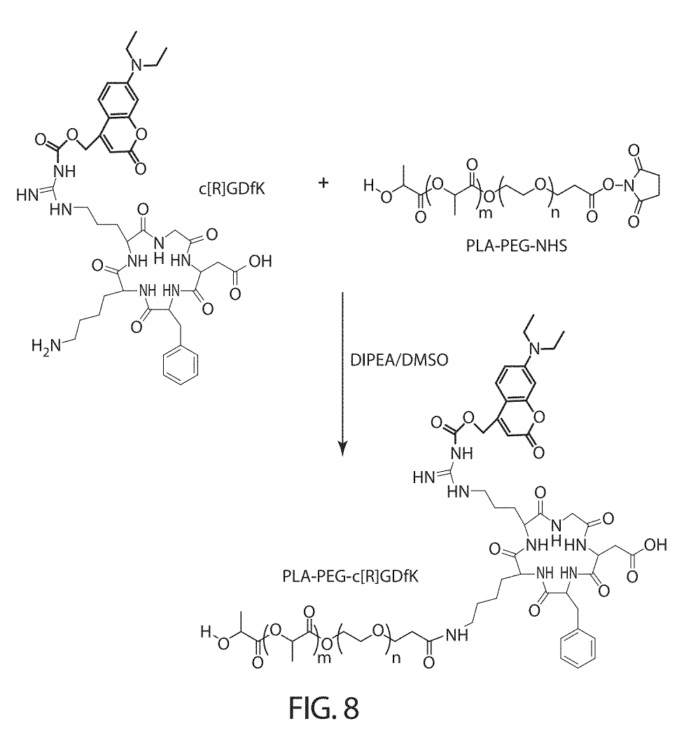

This example shows that the c[R]GDfK was conjugated onto block copolymer PLA-PEG to produce PLA-PEG-c[R]GDfK (FIG. 8). Photo-targeted polymeric micelles were made by the thin-film hydration method from PLA-PEG-c[R]GDfK and PLA-methoxy PEG (mPEG) (1:4 weight ratio). The resulting micellar nanoparticles, NP-c[R]GDfK, were dispersible in aqueous solution and had a hydrodynamic diameter of 33.0 nm.

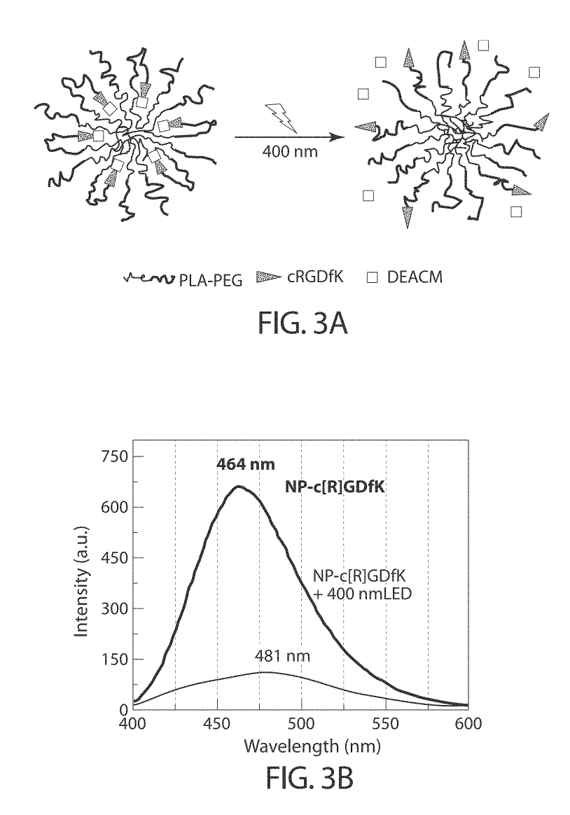

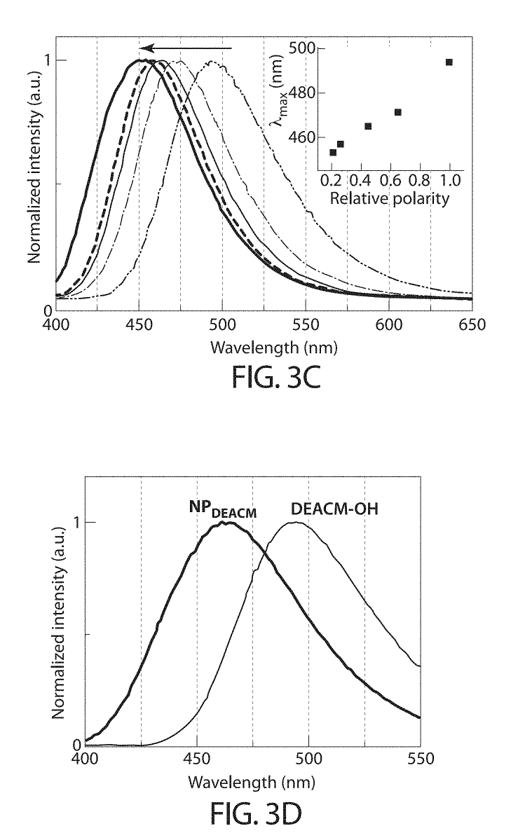

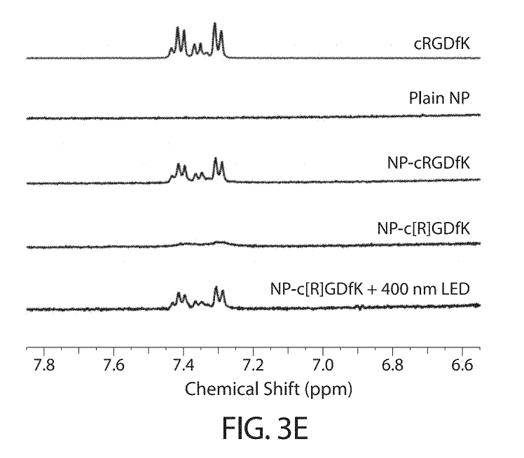

It is believed that the hydrophobicity of DEACM would cause it to localize in the PLA core of NP-c[R]GDfK (FIG. 3A). This was supported by the fact that the fluorescence spectrum of NP-c[R]GDfK (maximum at 464 nm; FIG. 3B) was blue-shifted in relation to that of free c[R]GDfK in aqueous solution (maximum at 493 nm; FIG. 2B), suggesting a change in ambient polarity. This possibility was supported by the finding that the fluorescence spectra of c[R]GDfK showed a clear blue-shift with decreasing solvent polarity (FIG. 3C). These data indicated that incorporation of DEACM into NP-c[R]GDfK placed it in a less polar environment than that of c[R]GDfK molecules in aqueous solution, i.e. that the DEACM was not on or in the hydrophilic PEG shell of the micelle, but that the PEG block had looped around so that the hydrophobic DEACM was in the less polar hydrophobic PLA core (FIG. 3A).

According to the suggested structural arrangement, irradiation of NP-c[R]GDfK with 400 nm LED light would release free DEACM-OH, which was more hydrophilic than the conjugated DEACM, into the aqueous environment (FIG. 3A). This was supported by a red-shift and decrease in the emission intensity of NP-c[R]GDfK solution upon irradiation (FIG. 3B). The red-shift was attributable to the increased polarity of DEACM's environment and the decrease in intensity to the quenching of fluorescence by water.

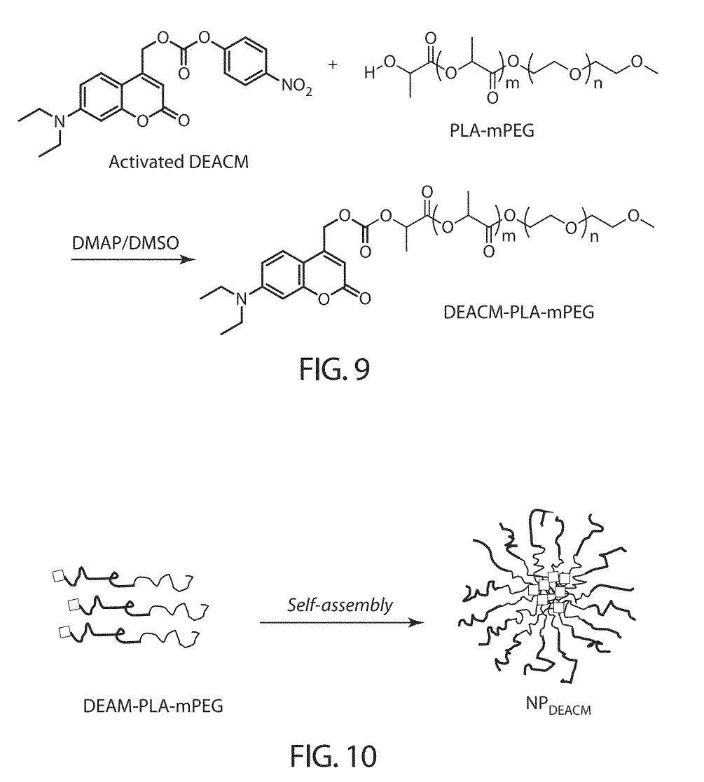

To further demonstrate that DEACM was localized in the hydrophobic core, DEACM-PLA-mPEG was synthesized (FIG. 9), which self-assembled into micellar nanoparticles (NP_{DEACM}; FIG. 10). Because the DEACM group was on the hydrophobic end of the conjugate, it should be located in the PLA core. The emission peak of NP_{DEACM} at 464 nm in PBS (FIG. 3D) further supports the view that the DEACM in NP-c[R]GDfK (FIG. 3B; emission peak also at 464 nm) was located in the hydrophobic PLA core. Moreover, the difference in the emission peaks of DEACM (FIG. 3D) from 464 nm (for NP_{DEACM}) to 494 nm (for DEACM-OH solution) is consistent with the change in the polarity of DEACM's environment from the nonpolar PLA core to aqueous conditions. These results indicate that DEACM was located in the PLA core of NP-c[R]GDfK.

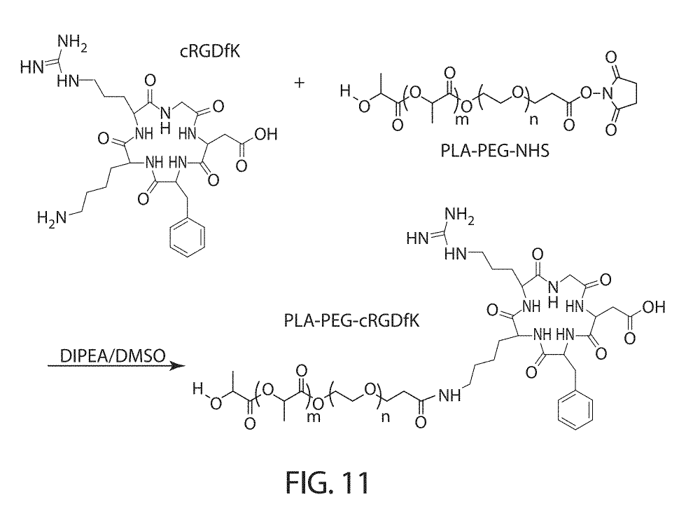

The structure discussed above was investigated directly by proton nuclear magnetic resonance (¹H NMR) spectroscopy (FIG. 3E). The ¹H NMR spectrum of NP-cRGDfK, which was formed with PLA-PEG-cRGDfK (FIG. 11) and PLA-mPEG (1:4 weight ratio), in D₂O showed chemical shifts of 7.25 to 7.45 ppm that were from the resonances of the phenyl protons of cRGDfK (FIG. 3E). Micelles formed with PLA-mPEG only (termed plain NP), did not show those peaks. The ¹H NMR spectrum of NP-c[R]GDfK also did not show those peaks, presumably because of the restricted mobility of the phenyl protons of cRGDfK within the PLA cores of the micelles, where they were because of DEACM's hydrophobicity. Irradiation at 400 nm resulted in the return of peaks at the same positions as in NP-cRGDfK. These results confirm that the phenylalanine in cRGDfK was located in the PLA core of NP-c[R]GDfK, and that photocleavage would return it to the surface. The absence of the characteristic peaks of DEACM-OH in FIG. 3E (NP-c[R]GDfK+400 nm LED group) may be because the released DEACM-OH is present at too low concentration to be detected by ¹H NMR. In addition, it is possible that some DCEAM-OH might have remained within the particle core after irradiation.

FIG. 3 shows c[R]GDfK being located in the hydrophobic core of NP-c[R]GDfK. FIG. 3A is a schematic of light-triggered activation of c[R]GDfK on NP-c[R]GDfK. FIG. 3B shows a fluorescence emission spectra of NP-c[R]GDfK and NP-c[R]GDfK irradiated for 1 min (50 mW cm⁻², 400 nm) in PBS. FIG. 3C shows fluorescence emission spectra of c[R]GDfK in different solvents, including tetrahydrofuran (THF, polarity relative to water: 0.21), chloroform (CHCl₃, 0.26), dimethyl sulfoxide (DMSO, 0.44), ethanol (0.65), and water (H₂O, 1.00). The arrow indicates the direction of decreasing solvent polarity. The inset is the plot of the emission maximum (λₘₐₓ) vs the relative polarity of solvents. FIG. 3D shows fluorescence emission spectra of NP_{DEACM} (micelles with DEACM on the hydrophobic end of the block polymer) and DEACM-OH in PBS. In FIG. 3C and FIG. 3D, the spectra were normalized so that their maximum intensities equaled. The excitation wavelength of all fluorescence measurements was 385 nm. FIG. 3E shows ¹H NMR spectra of free cRGDfK and different polymeric micelles in D₂O. Irradiation was performed with a 400 nm LED (50 mW cm⁻², 1 min).

FIG. 9 shows synthesis of DEACM-PLA-mPEG conjugate. Activated DEACM: (7-(diethylamino)-2-oxo-2H-chromen-4-yl)methyl (4-nitrophenyl) carbonate. DMAP: 4-Dimethylaminopyridine. DMSO: dimethyl sulfoxide. FIG. 10 shows a scheme of NP_{DEACM} self-assembled from the conjugate, DEACM-PLA-mPEG. The Z-average diameter for the intensity-weighted size distribution of N_{PDEACM} in PBS, determined by DLS, was 26.3+/−0.8 nm with a polydispersity of 0.050+/−0.030 (means+/−SD; n=4). The concentration of the micelles was 5.0 mg mL⁻¹. FIG. 11 shows synthesis of PLA-PEG-cRGDfK conjugate, differing from PLA-PEG-c[R]GDfK in not having the DEACM group. DIPEA: N,N-diisopropylethylamine. DMSO: dimethyl sulfoxide.

### Example 4

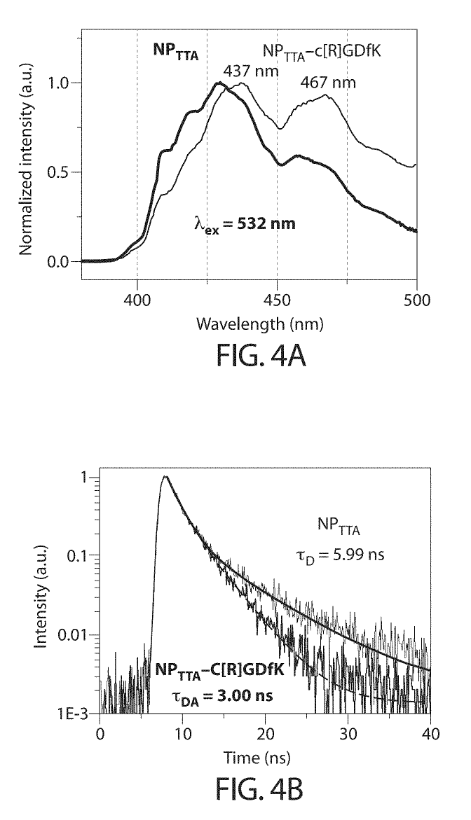

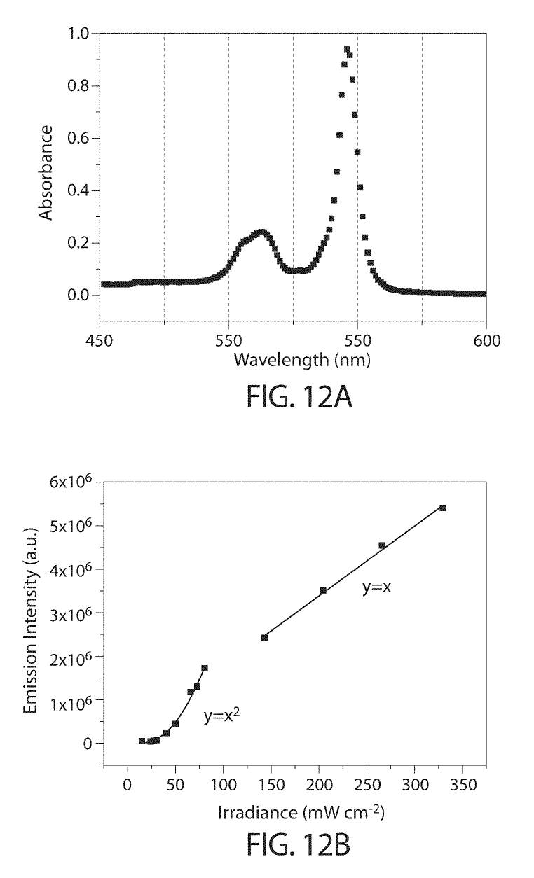

This example illustrates photocleavage triggered by TTA-UC, in accordance with one embodiment of the invention. The photosensitizer PdOEP and the annihilator DPA were incorporated into PLA-mPEG micellar nanoparticles (termed NP_{TTA}) by simple mixing during micelle formation. NP_{TTA} produced upconversion emission (FIG. 4A) under irradiation at 532 nm (commercially available green lasers; FIG. 12A). At low irradiances, the emission intensity of NP_{TTA} was proportional to the square of the irradiance, and linear at high irradiances (FIG. 12B), a pattern characteristic of TTA-UC. The TTA-UC efficiency in NP_{TTA} (see below) was 3.8% when irradiated at 532 nm and 150 mW cm⁻². Here, calculation of UC efficiency included multiplication by a factor of two to reflect the fact that emission of a single photon required the absorption of two.

Photo-targeted micellar nanoparticles containing PdOEP and DPA (termed NP_{TTA}-c[12] GDfK) with a hydrodynamic diameter of 36.7 nm were produced by self-assembly of PLA-PEG-c[R]GDfK and PLA-mPEG (1:4 weight ratio) together with PdOEP and DPA. The analyses of TTA-UC emission spectra and fluorescence lifetimes of DPA (FIG. 4) were consistent with FRET between dPA and DEACM in NP_{TTA}-c[R]GDfK. When irradiated with a 532 nm laser, the TTA-UC emission spectrum of NP_{TTA}-c[R]GDfK showed two peaks in the relatively long wavelengths at 437 and 467 nm, that were not present in NP_{TTA} (FIG. 4A), indicating that DEACM accepted the TTA-UC energy and emitted fluorescence. The peak at 467 nm could be attributed to the fluorescence of DEACM excited through FRET, because the fluorescence maximum of DEACM inside NP-c[R]GDfK was around 464 nm (FIG. 3B).

These changes in the spectrum of NP_{TTA} were not seen with a mixture of NP_{TTA} and free c[R]GDfK in solution, indicating that FRET did not happen, presumably since DEACM was far from the DPA (>10 nm) due to the separation of the PEG (MW 3000) shell (around 10 nm in thickness) and the free movement of c[R]GDfK molecules in solution.

In general, FRET reduces the fluorescence lifetime of donor fluorophores. In the absence of the acceptor DEACM, the fluorescence lifetime of the donor DPA(τ_{D}, tau-D) in NP_{TTA} was 5.99+/−0.04 ns (FIG. 4B); in the presence of the acceptor DEACM, the fluorescence lifetime of the donor DPA (τ_{DA}, tau-DA) in NP_{TTA}-c[R]GDfK was reduced to 3.00+/−0.02 ns, indicating the existence of FRET from DPA to DEACM. The FRET efficiency (E) was 49.9%, determined according to the following equation:

E=1−τ_{DA}/τ_{D}

where E indicates the percentage of excitation photons that contribute to FRET. These results demonstrate the occurrence of FRET in NP_{TTA}-c[R]GDfK from DPA to DEACM, as illustrated in FIG. 1B. Although a role for reabsorption in the energy transfer from DPA to DEACM cannot be ruled out, FRET played the major part, given that transfer by reabsorption is orders of magnitude less efficient than FRET.

FIG. 4 shows characterization of the FRET process in NP_{TTA}-c[R]GDfK. FIG. 4A shows TTA-UC emission spectra of NP_{TTA} and NP_{TTA}-c[R]GDfK when excited at 532 nm. The spectra were normalized so that their maximum intensities equalled 1. FIG. 4B shows decay of fluorescence of DPA in NP_{TTA} and NP_{TTA}-c[R]GDfK with excitation at 379 nm and emission at 410 nm. τ (tau) is fluorescence lifetime; τ_{DA} (tau-DA) is the lifetime in the presence of the acceptor DEACM; τ_{D} (tau-D) is the lifetime in the absence of the acceptor DEACM.

FIG. 12 shows spectroscopy of NP_{TTA}. FIG. 12A shows the absorption spectrum of PdOEP in toluene. FIG. 12B shows integrated emission intensity from NP_{TTA} plotted as a function of incident laser (532 nm) irradiance. The solid line is the best quadratic fit (χ², chi²) and linear fit (χ, chi) to the emission data. The wavelength of 532 nm was chosen based on the absorption of PdOEP and commercially available lasers. a.u.=arbitrary units.

### Example 5

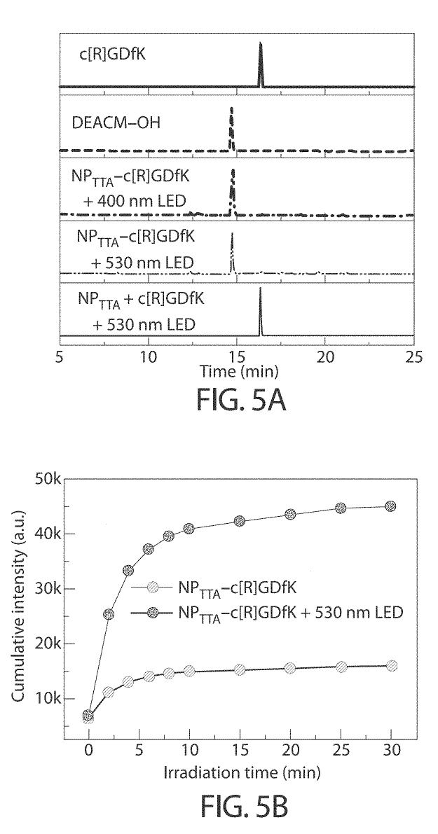

In this example, photocleavage of DEACM from NP_{TTA}-c[R]GDfK was assessed by irradiating the micelles with a 530 nm LED in PBS, separating the free DEACM-OH from the micelles by centrifugal filtration (50,000 Da cut-off), and analysing the filtrate by HPLC. The filtrate showed a peak with the same elution time as that of DEACM-OH and as the peak from the filtrate of NP_{TTA}-c[R]GDfK irradiated with a 400 nm LED (FIG. 5A). However, irradiation of a mixture of NP_{TTA} and free c[R]GDfK with a 530 nm LED did not cleave DEACM from c[R]GDfK; only a peak with the same elution time as free c[R]GDfK could be observed. These results showed that the photocleavage reaction occurring in NP_{TTA}-c[12] GDfK was mainly induced by TTA-UC through FRET.

The time course of photorelease of DEACM from NP_{TTA}-c[R]GDfK under continuous irradiation (FIG. 5B), assessed by measuring the fluorescence of the filtrates, showed that 5 min of irradiation released around 75% of DEACM from NP_{TTA}-c[R]GDfK. In contrast, the filtrate of the non-irradiated NP_{TTA}-c[R]GDfK showed relatively minimal release.

When NP-c[R]GDfK containing the photosensitizer PdOEP (but no DPA), termed NP_{PdOEP}-c[R]GDfK, were irradiated at 530 nm, DEACM was not cleaved from the micelles, which confirms that the photosensitizer alone could not transfer its energy to DEACM to cause photocleavage and 530 nm light irradiation could not directly cause photocleavage too. DPA does not absorb at 530 nm, so DPA alone could not have transferred the light energy to DEACM under irradiation at 530 nm.

FIG. 5 shows photocleavage of DEACM from NP_{TTA}-c[R]GDfK by TTA-UC. FIG. 5A shows HPLC traces (detected at 390 nm) demonstrating photocleavage. For the bottom three traces, it was only the filtrates of the samples (i.e. not the micelles) that were tested by HPLC. FIG. 5B shows cumulative fluorescent intensity (from integrated area under emission spectra; arbitrary units) of DEACM-OH photoreleased from NP_{TTA}-c[R]GDfK with 530 nm LED irradiation (150 mW cm⁻²) and in the dark.

### Example 6

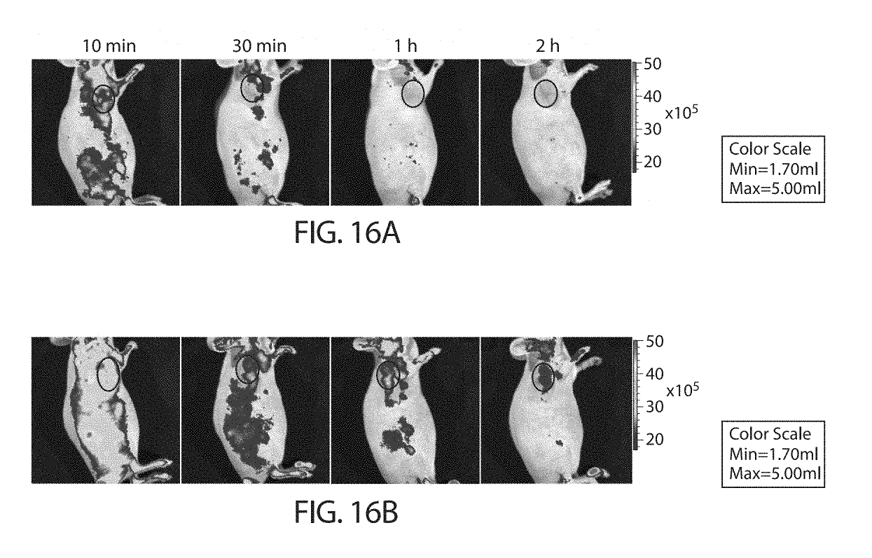

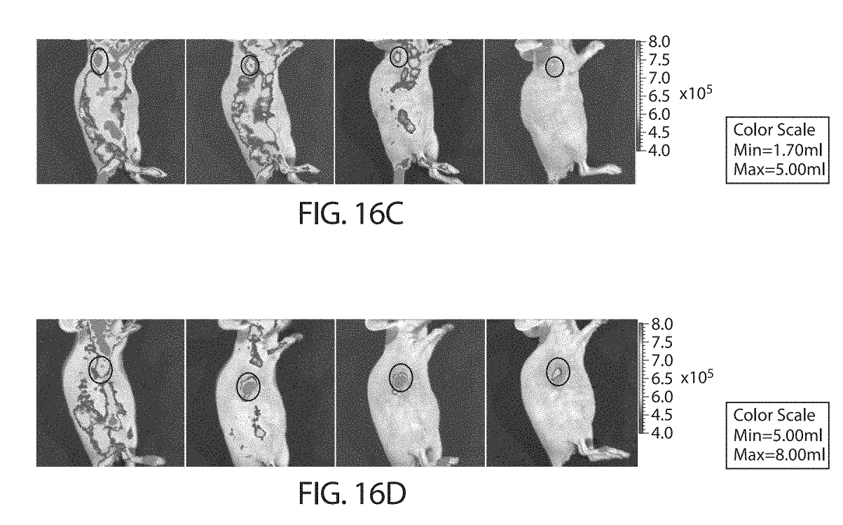

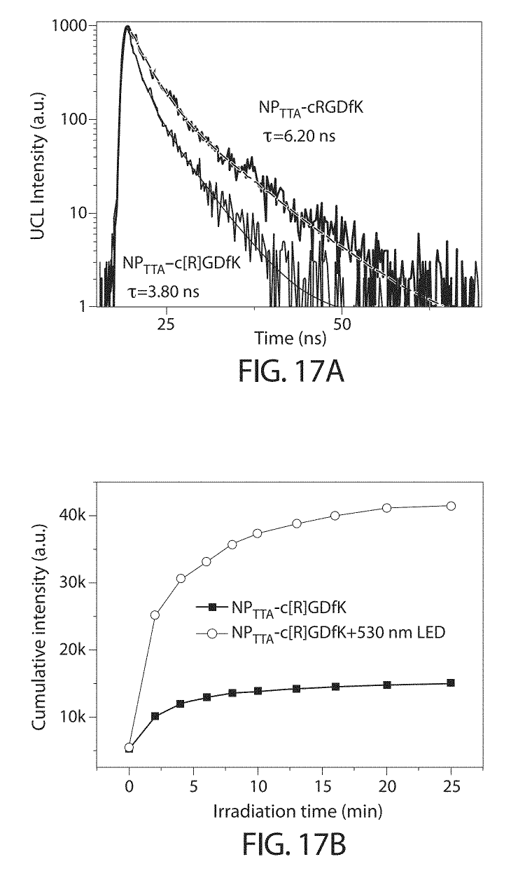

This example illustrates cell binding triggered by TTA-UC. Photo-triggered binding of NP_{TTA}-c[R]GDfK to cells by flow cytometry and confocal microscopy, was studied with micelles in which a hydrophilic dye, Lissamine™ rhodamine B (LRB), was covalently bound to the PLA-PEG copolymer (forming PLA-PEG-LRB; FIG. 16). LRB is photostable under irradiation with a 530 nm LED (150 mW cm⁻²) for at least 10 min (FIG. 17).

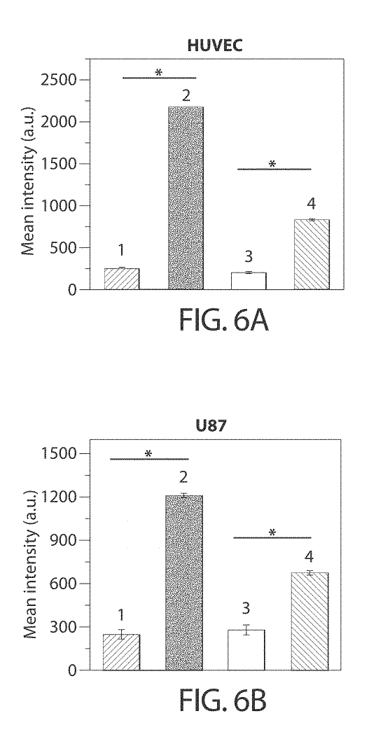

Human umbilical vein endothelial cells (HUVECs) and human glioblastoma (U87) cells, both of which express integrins including αᵥβ₃ integrin (alpha-V-beta-3), were incubated for 30 min with the following micellar nanoparticles containing 10% PLA-PEG-LRB: NP_{TTA}-cRGDfK, NP_{TTA}, NP_{TTA}-c[R]GDfK, and NP_{TTA}-c[R]GDfK, the latter only irradiated with a 530 nm LED (150 mW cm⁻², 5 min). Cell-associated LRB fluorescence (a measure of particle binding) was measured by flow cytometry (FIG. 6).

HUVECs incubated with NP_{TTA}-cRGDfK exhibited 7.8-fold greater fluorescence than those exposed to ligand-missing NP_{TTA} (FIG. 6A), indicating the ability of cRGDfK to target micelles to cells. NP_{TTA}-c[R]GDfK exhibited little binding to cells, showing that the caging group prevented ligand-mediated micelle binding to cells. Irradiation with a 530 nm LED (150 mW cm⁻², 5 min) increased cell binding of NP_{TTA}-c[R]GDfK by 3.3-fold. Similar results were obtained with U87 cells (FIG. 6B). The results indicated that the DEACM caging group was cleaved from NP_{TTA}-c[R]GDfK by TTA-UC energy, revealing the cRGDfK on the micelle surface and allowing micelle binding to cells. The cell-associated fluorescence of HUVECs and U87 cells irradiated while incubated with NP_{TTA}-c[R]GDfK was less than that of cells incubated with NP_{TTA}-cRGDfK (FIG. 6). This difference may be attributable to the fact that both particle types contained the same percentage (w/w) of PLA-PEG-c[R]GDfK or PLA-PEG-cRGDfK, but that, with PLA-PEG-c[R]GDfK, only 54.5% of polymers bore the peptide, while with PLA-PEG-cRGDfK, 96.6% bore the peptide.

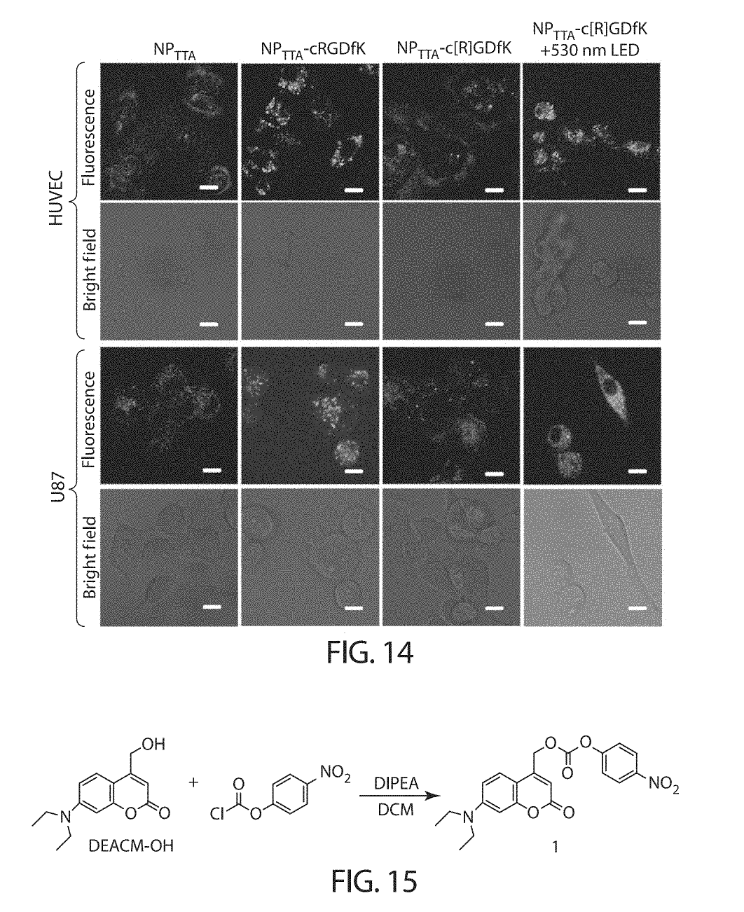

Light-controlled micelle binding was further confirmed by confocal laser scanning microscopy (CLSM) (FIG. 14). Irradiation with 530 nm LED (150 mW cm⁻², 5 min) induced cell binding and uptake of NP_{TTA}-c[R]GDfK in both HUVECs and U87 cells, while there was negligible binding and uptake of NP_{TTA} and non-irradiated NP_{TTA}-c[R]GDfK.

These examples generally demonstrate a photo-triggered targeting system using TTA-UC, by loading a photosensitizer (PdOEP) and annihilator (DPA) into PLA-PEG polymeric micelles functionalized with DEACM-caged cRGDfK. Due to its hydrophobicity, the DEACM caging group was enclosed in the hydrophobic PLA core, so that the distance between DPA (donor) and DEACM (acceptor) was short, allowing FRET. Cell binding of this nanoparticle system was enabled by a short exposure (5 min) to a relatively low irradiance by a green light LED at 150 mW cm⁻². In contrast to other upconversion-based approaches where coherent light is required, TTA-UC can be triggered with noncoherent LED light.

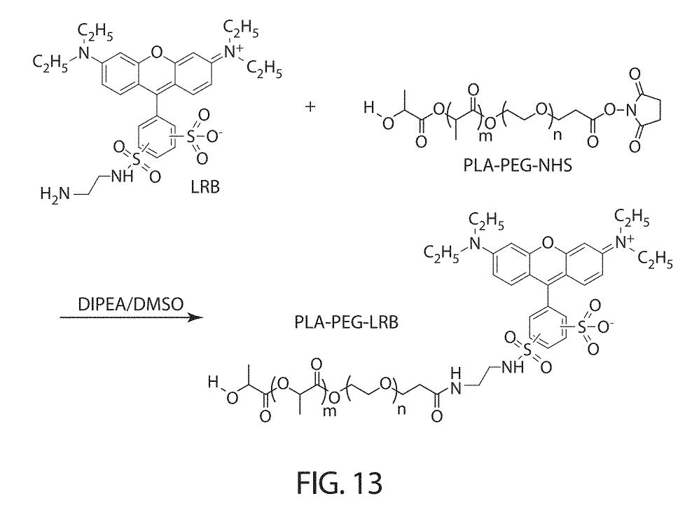

FIGS. 6A-6B illustrate flow cytometric analysis of cell binding and uptake of micelles. In these figures, (1) NP_{TTA}, (2) NP_{TTA}-cRGDfK, (3) NP_{TTA}-c[R]GDfK, and (4) NP_{TTA}-c[R]GDfK irradiated with a 530 nm LED (150 mW cm⁻², 5 min) were incubated with HUVECs (FIG. 6A) or U87 cells (FIG. 6B) at 37° C. for 30 min. Cell fluorescence was then measured by flow cytometry. Data are mean+/−SD (n=4), *p<0.001. All micelles were labelled with LRB. FIG. 13 shows synthesis of PLA-PEG-LRB conjugate. LRB: Lissamine™ rhodamine B ethylenediamine. DIPEA: N,N-diisopropylethylamine. DMSO: dimethyl sulfoxide. FIG. 14 shows confocal microscopic analysis of cell binding and uptake of nanoparticles. Confocal laser scanning microscopy and bright field images of HUVECs and U87 cells which were incubated at 37° C. for 30 min with NP_{TTA}, NP_{TTA}-cRGDfK, NP_{TTA}-c[R]GDfK, and NP_{TTA}-c[R]GDfK, the latter irradiated with a 530 nm LED (150 mW cm⁻², 5 min prior to the 30 min incubation). All nanoparticles were labelled with LRB. The scale bar is 10 micrometers. Images are representative of two independent experiments.

### Example 7

Following is additional description of materials and methods used in the above examples. Most chemicals were purchased from Sigma-Aldrich (Missouri, USA) and used without further purification unless otherwise stated. Poly(D,L-lactic acid)(2000)-poly(ethylene oxide)(3000)-N-hydroxysuccinimide (PLA-PEG-NHS) and PLA(2000)-methoxy PEG (mPEG, 2000) (PLA-mPEG) were ordered from Advanced Polymer Materials (Montreal, Canada). 7-diethylamino-4-hydroxymethylcoumarin was bought from INDOFINE Chemical Company (New Jersey, USA). Lissamine™ rhodamine B ethylenediamine (LRB) and cyclo-(RGDfK) were purchased from AnaSpec (California, USA). c[R]GDfK was custom made from AnaSpec from (7-(diethylamino)-2-oxo-2H-chromen-4-yl)methyl (4-nitrophenyl) carbonate. Human umbilical vein endothelial cells (HUVECs) and endothelial cell growth media kits (EGM™-2 BulletKit, Catalog No. CC-3162) were purchased from Lonza (New Jersey, USA). Human glioblastoma (U87) cells were purchased from American Type Culture Collection (ATCC) (Virginia, USA) The CellTiter 96® AQueous One Solution Cell Proliferation Assay solution was purchased from Promega (Medison, USA). Other cell culture agents were purchased from Life Technologies (Now York, USA).

Synthesis and characterization of (7-(diethylamino)-2-oxo-2H-chromen-4-yl)methyl (4-nitrophenyl)carbonate is shown in FIG. 15. 7-diethylamino-4-hydroxymethylcoumarin (DEACM-OH, 150 mg, 0.606 mmol) was dissolved in 22 mL of dichloromethane (DCM). N,N-diisopropylethylamine (DIPEA, 1.055 mL, 6.06 mmol) and 4-nitrophenyl chloroformate (1.22 g, 6.06 mmol) were then added into the above solution on an ice bath. After stirring for 15 min, the mixture was allowed to warm to room temperature and stirred for 2 h. The reaction solution was washed with 0.01 M aqueous HCl (125 mL×2), and then the organic layer was collected and dried with anhydrous MgSO₄. The dried organic solution was filtered by a filter funnel and concentrated with a rotary evaporator. The crude product was purified by flash chromatography (CombiFlash® system, Teledyne ISCO, Nebraska, USA) on a normal-phase silica flash column (RediSep Rf, Teledyne ISCO, Nebraska, USA) using DCM and methanol as mobile phase.

Photocleavage of c[R]GDfK molecule. A disposable macro fluorescence cuvette containing 400 microliters of c[R]GDfK PBS solution (50 microgram/mL) was irradiated under an 11-mm LED (400 nm) collimator with a Multi-channel Universal LED controller (Mightex Systems, CA, USA). The temperature of the solution was controlled at 37° C. in a t50/Eclipse cuvette holder with a TC 125 temperature controller (Quantum Northwest, WA, USA). The LED irradiance was measured with a PM100USB Power and Energy Meter (ThorLabs, NJ, USA). At each irradiation time point, 50 microliters of the irradiated solution was analyzed by reverse-phase high-performance liquid chromatography (RP-HPLC) with an Agilent 1260 Infinity LC system and a Poroshell 120 EC-C18 column (4.6×100 mm) (Agilent Technologies, CA, USA). The detection wavelength was 390 nm.

Synthesis of polymer conjugates. To synthesize PLA-PEG-c[R]GDfK, 14 mg of PLA-PEG-NHS and 3 mg of c[R]GDfK were dissolved in 200 microliters of dimethyl sulfoxide (DMSO). After 0.6 microliters of DIPEA was added, the mixture was shaken for 5 hours at room temperature. The mixture was then dialyzed against 4 changes of 5 L of distilled water with a Spectra/Por® 6 dialysis membrane (molecular weight cut-off, MWCO: 3.5 kD) at 4° C. After 2 days of dialysis, the dialyzed solution was freeze-dried to get the solid product. The same procedure was used to synthesize PLA-PEG-LRB and PLA-PEG-cRGDfK. For DEACM-PLA-mPEG, the same procedure was used expect the use of 4-dimethylaminopyridine (DMAP) instead of DIPEA. The conjugation efficiency of all of the polymer conjugates measured by ¹H NMR was more than 90% except for that of PLA-PEG-c[R]GDfK (54.4%).

Preparation of polymeric micelles. To prepare NP-c[R]GDfK, PLA-PEG-c[R]GDfK (2.0 mg) and PLA-mPEG (8.0 mg) were co-dissolved in 5 mL of acetonitrile. Rotary evaporation was used to slowly evaporate the solvent at 45° C. The dried polymer film was hydrated with 2 mL of PBS at 60° C. For other polymeric micelles, the same procedure was used except that different compounds were added for each micelle, plain NP: PLA-mPEG (10.0 mg); NP_{TTA}-cRGDfK: PLA-PEG-cRGDfK (2.0 mg) and PLA-mPEG (8.0 mg).

To prepare NP_{TTA}-c[R]GDfK, PLA-PEG-c[R]GDfK (2.0 mg) and PLA-mPEG (8.0 mg) were co-dissolved in 5 mL of acetonitrile, then mixed with 0.5 mL of DPA (2.0 mM) and 0.05 mL of PdOEP (0.2 mM) toluene solutions. Rotary evaporation was used to slowly evaporate the solvents at 45° C. The dried polymer film was hydrated with 2 mL of PBS at 60° C. For other TTA-containing micelles, the same procedure was used except the use of different composition of polymers for each micelles, NP_{TTA}: PLA-mPEG (10.0 mg); NP_{TTA}-cRGDfK: PLA-PEG-cRGDfK (2.0 mg) and PLA-mPEG (8.0 mg); LRB-labelled NP_{TTA}: PLA-PEG-LRB (1.0 mg) and PLA-mPEG (9.0 mg); LRB-labelled NP_{TTA}-cRGDfK: PLA-PEG-cRGDfK (2.0 mg), PLA-PEG-LRB (1.0 mg) and PLA-mPEG (7.0 mg); LRB-labelled NP_{TTA}-c[R]GDfK: PLA-PEG-c[R]GDfK (2.0 mg), PLA-PEG-LRB (1.0 mg) and PLA-mPEG (7.0 mg).

Loading efficiency of DPA and PdOEP in NP_{TTA}. The NP_{TTA} was filtered through a 200 nm filter membrane to remove aggregated un-encapsulated PdOEP and DPA. The NP_{TTA}-containing filtrate was then mixed with acetonitrile. The UV-visible absorption spectrum of the mixture was measured and compared to the standard curves for PdOEP and DPA. The loading efficiencies of PdOEP and DPA in NP_{TTA} were calculated to be 44.5+/−7.9% (n=4) and 69.1+/−8.6% (n=4), respectively. Based on those loading efficiencies, a molar ratio of 1:235 (PdOEP: DPA) was obtained within the micelles.

Transmission electron microscopy. A 10 microliter aliquot of the nanoparticle solution was deposited on a copper grid coated by a carbon film. After 2 min, excess solution was blotted by a filter paper. The sample was dried at room temperature and then imaged on a Tecnai G2 Spirit BioTWIN transmission electron microscope, operating at 80 kV.

Dynamic light scattering. The size of nanoparticles was measured with a Delsa Nano C particle analyzer (Beckman Coulter, CA, USA). Nanoparticle solution (100 microliters) was put into a disposable cuvette (Eppendorf UVette) and tested at 25° C. with the accumulation times of 70. Each sample was tested at least 3 times. The hydrodynamic diameter was calculated by averaging the repeated cumulate results of diameters.

Spectroscopic characterization. The upconversion luminescence emission spectra were recorded on Edinburgh FL-920 instrument. The excitation source used an external 0-500 mW adjustable 532 nm semiconductor laser (Changchun fs-optics Co., China) with an optic fiber accessory, instead of the Xeon source in the spectrophotometer. The fluorescence lifetime was measured on Edinburgh FL-920 instrument with the semiconductor laser as the excitation source (excitation wavelength 379 nm). The fluorescent spectra and UV-Vis spectra were recorded on an Agilent Cary Eclipse fluorescence spectrophotometer and an Agilent 8453 UV-Vis G1103A spectrophotometer.

Measurement of upconversion emission quantum efficiency. The upconversion emission quantum efficiency (Φ_{UC}, Phi-UC) of NP_{TTA} in water was determined according to the following equation, with rhodamine B in ethanol as a standard reference. The equation is multiplied by factor of two in order to make the maximum quantum yield to be unity:

$${Φ}_{UC} = 2 {Φ}_{std} ( \frac{{A}_{std}}{A} ) ( \frac{{I}_{UCL}}{{I}_{std}} ) {( \frac{η}{{η}_{std}} )}^{2}$$

where Φ_{UC} (Phi-UC) is the upconversion emission quantum yield of NP_{TTA} and Φ_{std} (Phi-std) is the fluorescence quantum efficiency of rhodamine B. A=absorbance of NP_{TTA}, and A_{std}=absorbance of hodamine B, respectively. I_{UCL}=integrated upconversion emission intensity of NP_{TTA} and I_{std}=integrated fluorescence intensity of rhodamine B. η (eta)=refractive index of water (for NP_{TTA}) and η_{std} (eta-std) is the refractive index of ethanol (for rhodamine B).

Lifetime analysis. A bi-exponential fit was employed. Both the fast and the slow components are presented in Table 1, as well as the fractional contributions of the fast and slow components. The lifetime was calculated according to the following equation. The photon-weighted average lifetime, as described by Lakowicz,

$$τ = \frac{{τ}_{fast}^{2} \times {P}_{fast} + {τ}_{slow}^{2} \times {P}_{slow}}{{τ}_{fast} \times {P}_{fast} + {τ}_{slow} \times {P}_{slow}}$$

Table 1 shows the lifetime decay fitting data of NP_{TTA}-c[R]GDfK and NP_{TTA}, λₑₓ=375 nm, λₑₘ=410 nm. τ_{fast} and P_{fast} represent the lifetime and the fractional contribution of the fast component of the decay, respectively. τ_{slow} and P_{slow} represent the lifetime and the fractional contribution of the slow component of the decay, respectively. τ is the photon-weighted average lifetime of DPA. χ² is the fit quality criterion.

TABLE 1

| | τ_{fast} | | τ_{slow} | | | |
|---|---|---|---|---|---|---|
| Sample | (ns) | P_{fast} | (ns) | P_{slow} | χ² | τ (ns) |
| NP_{TTA}-c[R]GDfK | 1.26 | 30.0% | 3.29 | 70.0% | 1.054 | 3.00 ± 0.02 |
| NP_{TTA} | 1.49 | 36.6% | 6.60 | 63.4% | 1.186 | 5.99 ± 0.04 |

Photocleavage of DEACM from NP_{TTA}-c[R]GDfK. One milliliter of NP_{TTA}-c[R]GDfK solution (1 mg mL⁻¹) was put in a quartz cuvette and irradiated with LED light (530 nm, 150 mW cm⁻²). At each irradiation time, the solution was put in an Amicon® Ultra centrifugal filter (50,000 Da cut-off) and centrifuged at 4000 rmp for 15 min. The fluorescence of the filtrate was measured by Cary Eclipse fluorescence spectrophotometer (Agilent, CA, USA).

Flow cytometry. Cells were cultured in cell growth media in a humidified atmosphere with 5% CO₂ at 37° C. For the cytometry testing, cells were seeded on 48-well plate at a density of 15,000 cells per well. After an overnight incubation, the growth media was replaced with the fresh media containing different nanoparticles at the concentration of 0.4 mg mL⁻¹: NP_{TTA}, NP_{TTA}-cRGDfK, NP_{TTA}-c[R]GDfK, and NP_{TTA}-c[R]GDfK with irradiation (530 nm, 150 mW cm⁻², 5 min). All nanoparticles were labelled with LRB. After 30 min of incubation at 37° C., the cells were washed with PBS twice and detached with 150 microliters of 0.25% Trypsin-EDTA solution. The cells were suspended with 350 microliters of trypsin neutralizing solution (TNS) and transferred into BD Falcon round-bottom tube (BD Bioscience, NJ, USA). The flow cytometry was run on BD LSR Fortessa cell analyzer (BD Bioscience, NJ, USA).

Confocal laser scanning microscopy. Cells were seeded on a 35-mm glass bottom dish with collagen coating (MatTek Corporation, MA, USA) at a density of 150,000 cells per well. After an overnight incubation, the growth media was replaced with the fresh media containing different naniparticles at the concentration of 0.4 mg mL⁻¹: NP_{TTA}, NP_{TTA}-cRGDfK, NP_{TTA}-c[R]GDfK, and NP_{TTA}-c[R]GDfK with irradiation (530 nm, 150 mW cm⁻², 5 min). All nanoparticles were labelled with LRB. After 30 min of incubation at 37° C., the cells were washed with PBS twice and imaged with Leica SPSX laser scanning confocal microscope (Laica Microsystems, IL, USA).

Statistics. All p values were calculated by the unpaired t-test using Origin 8.0 software (Massachusetts, USA). Data are mean+/−SD.

Energy Unit Conversion. The calculation for energy unit conversion from wavelength (nm) to electron volt (eV) is based on the Planck-Einstein relation:

E=hc/λ,

where E=energy (eV); h=Planck's constant=4.135667516×10⁻¹⁵ eV s; c=speed of light=299792458 m s⁻¹; λ (lambda)=light wavelength (nm).

### Example 8

This example illustrates phototargeting in vivo, in accordance with one set of embodiments. Tumor-bearing (human glioblastoma) nude mice received i.v. injection of NP_{TTA}-c[R]GDfK containing 10% PLA-PEG-LRB. After the injection, the tumors were irradiated with a 530 nm LED (200 mW cm⁻², for 5 min). The mice were imaged by an in vivo imaging system (IVIS) at 10 min, 30 min, 1 h, and 2 h after the injection. The control groups were injected with the following micellar nanoparticles containing 10% PLA-PEG-LRB: NP_{TTA}, NP_{TTA}-cRGDfK, NP_{TTA}-c[R]GDfK. The mouse injected with NP_{TTA}-cRGDfK showed greater fluorescence signal at the tumor site than the one with NP_{TTA} at 2 h after the injection (FIGS. 16A and 16B), indicating the ability of cRGDfK to target nanoparticles to tumors in vivo. Irradiation with a 530 nm LED (200 mW cm⁻², 5 min) increased fluorescence signal at the tumor site (FIGS. 16C and 16D, indicating the light can control the targeting of nanoparticles to tumors in vivo.

FIG. 16 shows IVIS images of tumor-bearing mice injected with (FIG. 16A) NP_{TTA}, (FIG. 16B) NP_{TTA}-cRGDfK, (FIG. 16C) NP_{TTA}-c[R]GDfK without irradiation on tumor site and (FIG. 16D) NP_{TTA}-c[R]GDfK with irradiation on tumor site. The oval indicates tumor sites. The injection volume is 150 microliters. The concentration of all of the nanoparticles is 5 mg mL⁻¹.

### Example 9

A large proportion of the payload delivered by nanoparticulate therapies is deposited not in the desired target destination, but in off-target locations such as the liver and spleen. This example demonstrates that phototargeting can improve the specific targeting of nanoparticles to tumors. The combination of efficient triplet-triplet annihilation upconversion (TTA-UC) and Förster resonance energy transfer (FRET) processes allowed in vivo phototargeting at a safe irradiance (200 mW/cm²) over a short period (5 min) using green light.

Some of the above examples describe an in vitro approach to targeting polymeric micelles to cells, whereby visible light was upconverted by triplet-triplet annihilation-based upconversion (TTA-UC) and the energy transferred by Förster resonance energy transfer (FRET) to cleave a covalent bond. The cleavage released a caging group from a ligand, allowing cell binding. This example investigates whether this system can be used in vivo to provide safe and rapid phototargeting at a low irradiance, and whether it can decrease off-target binding in liver and spleen and provide a higher specificity to tumors.

A photosensitizer, palladium octaethylporphyrin (PdOEP), annihilator 9,10-diphenylanthracene (DPA) and DiR (a NIR dye for in vivo imaging) were encapsulated in the hydrophobic core of polymeric micelles formed by self-assembly of a chemically modified poly(D,L-lactic acid)-poly(ethylene oxide) (PLA-PEG) block copolymer (FIG. 8). The targeting peptide, cRGDfK, was conjugated to the end of the PEG chain away from the PLA moiety. A photocleavable caging group [7-(diethylamino) coumarin-4-yl]methyl (DEACM) was conjugated to cRGDfK (termed c[R]GDfK) to block its binding to receptors. The resulting nanoparticles (termed NP_{TTA}-c[R]GDfK) had a hydrodynamic diameter of 59.0 nm.

The above examples show the fluorescence lifetime of DPA in NP_{TTA}-c[R]GDfK without DiR to demonstrate that FRET occurred from DPA (donor) to DEACM (acceptor). These experiments used a similar experiment with DiR-loaded NP_{TTA}-c[R]GDfK, to confirm FRET still occurred. In the absence of the acceptor DEACM (NP_{TTA}-cRGDfK: differed from NP_{TTA}-c[R]GDfK in not having the DEACM group (FIG. 11)), the lifetime of DPA was 6.20 ns (FIG. 17A); in the presence of DEACM (NP_{TTA}-c[R]GDfK), the lifetime of DPA was reduced to 3.80 ns, indicating the existence of FRET from DPA to DEACM in NP_{TTA}-c[R]GDfK containing DiR. The efficiency of FRET from DPA to DEACM in DiR-containing NP_{TTA}-c[R]GDfK was calculated to be 39% (FIG. 17A; see below for calculations), which is lower than that without DiR. The decrease of the FRET efficiency is probably because DiR loading increases the distance between DPA and DEACM in NP_{TTA}-c[R]GDfK. The FRET efficiency is inversely proportional to the sixth power of the distance between donor and acceptor, making FRET extremely sensitive to small changes in distance.

FIG. 17 shows FRET and photocleavage of NP_{TTA}-c[R]GDfK. FIG. 17A shows fluorescence decay of DPA in NP_{TTA}-c[R]GDfK and NP_{TTA}-cRGDfK with excitation at 379 nm and emission at 410 nm. τ (tau) denotes the fluorescence lifetime. FIG. 17B shows cumulative fluorescence intensity reflecting release of DEACM-OH from NP_{TTA}-c[R]GDfK with or without 530 nm LED irradiation (200 mW/cm²).

The time course of phototriggered release of DEACM-OH from NP_{TTA}-c[R]GDfK was assessed by measuring the fluorescence of release media (see below) under continuous irradiation (FIG. 17B). About 70% of DEACM-OH was released from NP_{TTA}-c[R]GDfK after irradiation at 200 mW/cm² for 5 min. Non-irradiated NP_{TTA}-c[R]GDfK showed minimal release of DEACM-OH (FIG. 17B).

Irradiation at 385 nm, a wavelength close to the maximal absorption wavelength (388 nm) of the caging group DEACM, could cleave DEACM from cRGDfK directly. The TTA-UC mechanism also allowed for the release of DEACM-OH by irradiation at 530 nm (FIG. 17B).

The cytotoxicity of irradiation itself was assessed at 385 nm and 530 nm in U87 cells, which was used in the in vivo cancer model below, by MTS assay. Green light (530 nm) irradiation for 5 min at up to 205 mW/cm² did not induce any significant cytotoxicity, while irradiation at 385 nm resulted in decreasing cell viability with increasing irradiance. These results suggested that 530 nm was safer than 385 nm light for phototargeting.

The cytotoxicity of various components of the nanoparticles was assessed in U87 cells. Free DEACM-OH showed no toxicity over three days of continuous exposure at concentrations up to 100 micromolar, much higher than likely to occur systemically in vivo. Nanoparticulate formulations containing PdOEP, DPA and DiR, including NP_{TTA}-c[R]GDfK, NP_{TTA}-cRGDfK and NP_{TTA} (did not have DEACM or the targeting peptide) showed minimal toxicity over 3 days at nanoparticle concentrations up to 1 mg/mL, much higher than likely to occur in vivo after systemic delivery. Irradiation (530 nm LED light, 200 mW/cm², 5 min) of NP_{TTA}-c[R]GDfK induced a slight increase (p<0.05 in comparison to NP_{TTA}-c[R]GDfK group at Day 3) in cytotoxicity (albeit at particle concentrations that are unlikely to occur except if injected directly into tissues).

### Example 10

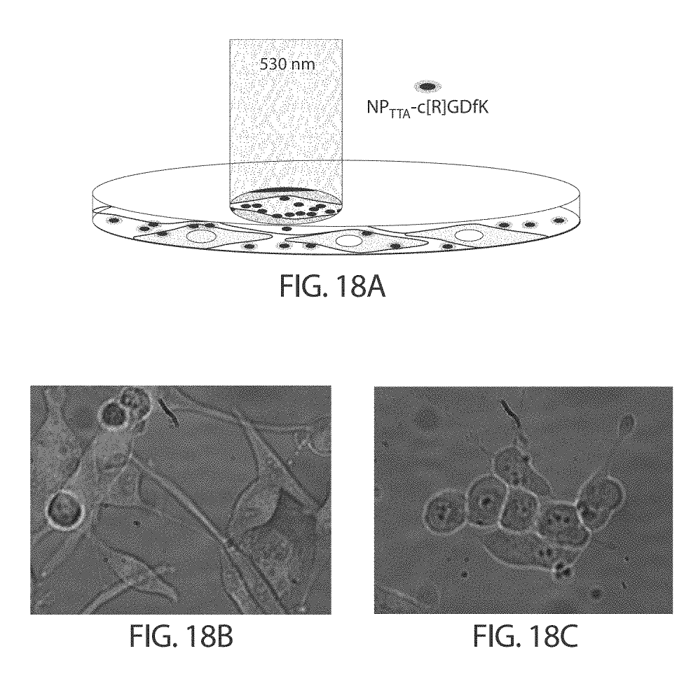

To demonstrate the phototargeting capability of NP_{TTA}-c[R]GDfK in vitro, U87 cells were incubated in this example with NP_{TTA}-c[R]GDfK labeled with the fluorescent probe Lissamine Rhodamine B (LRB; see below) which could be visualized by the microscopy system. An 8.5 cm culture dish was covered with an aluminum foil mask to prevent light from reaching the cells except through a 3 mm aperture (FIG. 18A), then irradiated (530 nm, 200 mW/cm², 5 min) and washed to remove unbound particles (see below). Fluorescence microscopy showed that particles bound to an area 20 mm in diameter beneath the hole in the mask; the large size of the area was probably due to diffusion of nanoparticles or light beam scattering. The average fluorescence intensity (calculated by Image J) of each cell in the light-exposed area (FIG. 18B) was ˜5 fold stronger than that in the area not exposed to light (FIG. 18C).

FIG. 18 shows in vitro phototargeting in U87 cells. FIG. 18A is a schematic illustration of the experimental setup for phototargeting in vitro. FIGS. 18B and 18C show representative mergers of fluorescence (LRB) and brightfield views of U87 cells in irradiated (FIG. 18B) and non-irradiated areas (FIG. 18C; ˜3.0 cm from the irradiated area) of cell culture.

### Example 11

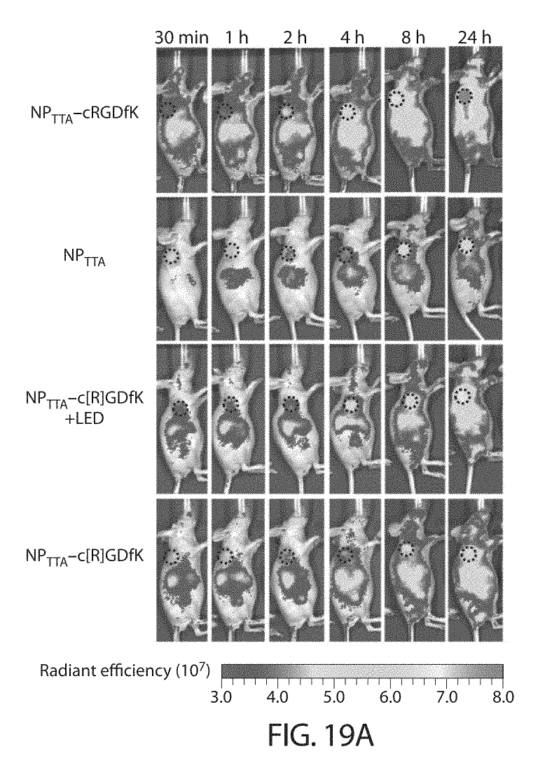

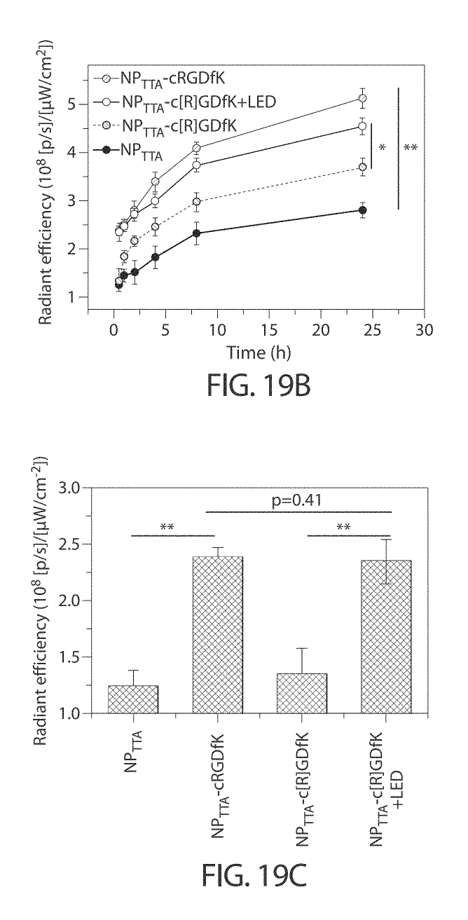

To assess tumor targeting in vivo, in this example, nude mice bearing subcutaneous ˜100-200 mm³ U87 glioblastomas on the right shoulder were administered intravenously 200 microliters of 5 mg/mL NP_{TTA}-cRGDfK, NP_{TTA}, NP_{TTA}-c[R]GDfK or NP_{TTA}-c[R]GDfK followed by irradiation of the tumor sites (530 nm LED, 200 mW/cm², 5 min; this group is abbreviated NP_{TTA}-c[R]GDfK+LED). They then underwent whole-body fluorescence imaging (FIG. 9A) at different time points with an in vivo imaging system (IVIS). The fluorescence intensity of tumors increased in a time-dependent manner in all groups (FIG. 19B). Groups with uncaged ligand (NP_{TTA}-c[R]GDfK+LED and NP_{TTA}-cRGDfK) showed stronger fluorescence than those without (non-irradiated NP_{TTA}-c[R]GDfK and NP_{TTA} groups) at all time points. In the NP_{TTA}-c[R]GDfK+LED group, fluorescence at the tumor site 30 minutes after injection was similar to that in the uncaged NP_{TTA}-cRGDfK group (p=0.41), and was enhanced 1.8-fold compared to that in the group receiving NP_{TTA}-c[R]GDfK without irradiation (P<0.005) (FIG. 19C), suggesting successful ligand uncaging and target binding. Irradiation did not enhance the intratumoral fluorescence intensity of NP_{TTA}, suggesting that targeting was not due to a direct effect of the irradiation itself such as enhanced capillary permeability and that therefore the enhanced accumulation of NP_{TTA}-c[R]GDfK in tumors by irradiation was due to photoactivation of c[R]GDfK in NP_{TTA}-c[R]GDfK. These results are consistent with the cRGDfK group in NP_{TTA}-c[R]GDfK being deactivated by DEACM and activated upon 530 nm irradiation, allowing ligand binding.

FIG. 19 shows the effect of light triggering on the biodistribution of DiR fluorescently labeled nanoparticles. FIG. 19A shows representative whole-body fluorescence images of subcutaneous U87 tumor-bearing mice intravenously injected with nanoparticles without or with subsequent irradiation at the tumor site (530 nm light, 200 mW/cm², 5 min). Tumors are indicated by dashed circles. FIG. 19B shows the time course of intratumoral fluorescence in FIG. 19A over 24 h. FIG. 19C shows intratumoral fluorescence in FIG. 19A 30 min after injection. Data are means+/−SD (n=4). *p<0.05, **p<0.005.

### Example 12

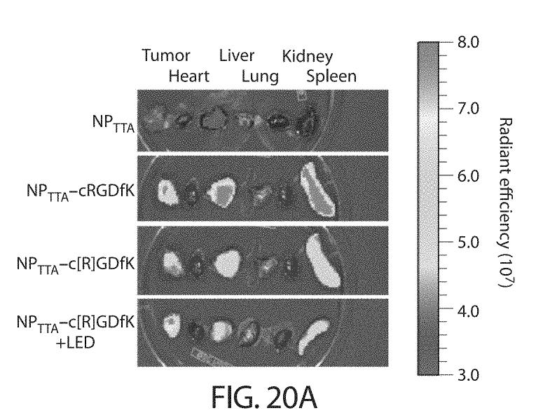

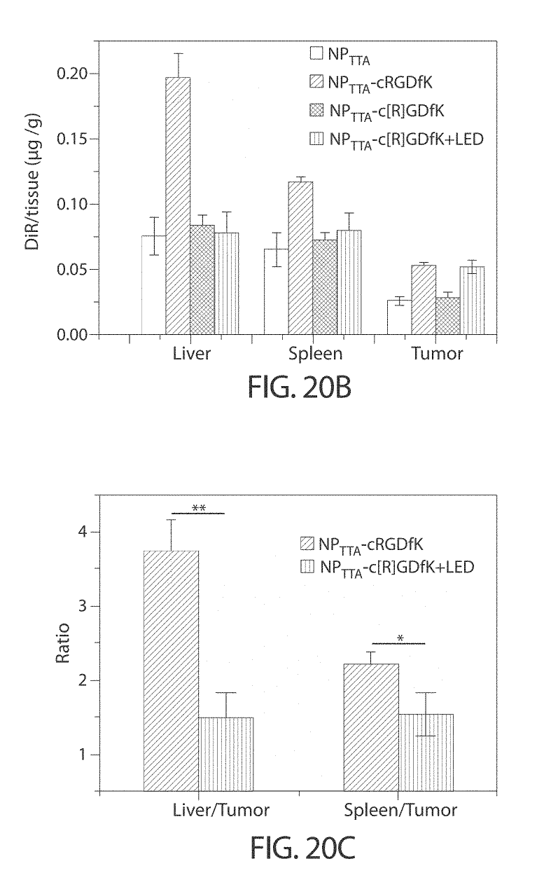

Biodistribution of nanoparticles was assessed in this example by measuring the fluorescence of DiR in various harvested organs and tumors upon necropsy 24 h after injection (FIG. 20A), using an IVIS system. The fluorescence intensity of tumors in the NP_{TTA}-c[R]GDfK+LED group was similar to that in non-irradiated animals administered NP_{TTA}-cRGDfK, indicating that ligand uncaging led to effective target binding. However, the fluorescence intensity in the liver and spleen were lower in the NP_{TTA}-c[R]GDfK+LED group than in the NP_{TTA}-cRGDfK group.

As a confirmatory quantitative test, DiR was extracted from the same organs and tumors, harvested 24 h after injection, and the content (micrograms of DiR/g of tissue) was measured by fluorometry (FIGS. 20B and 20C). The mean tumor content of DiR in the NP_{TTA}-c[R]GDfK+LED group was 1.8-fold and 1.9-fold higher than those in non-irradiated NP_{TTA}-c[R]GDfK and NP_{TTA} groups (p<0.001 for both), respectively, and was comparable to that in NP_{TTA}-cRGDfK (p=0.38). These data confirmed that irradiation successfully uncaged the targeting peptide. In liver and spleen, the DiR content in the NP_{TTA}-c[R]GDfK+LED group was similar to that in non-irradiated NP_{TTA}-c[R]GDfK, but lower than that in the NP_{TTA}-cRGDfK group (p<0.01 in both liver and spleen). The mean liver-to-tumor and spleen-to-tumor ratios of DiR content in the NP_{TTA}-c[R]GDfK+LED group were 2.5-fold (p<0.005) and 1.5-fold (p<0.05) lower than in the NP_{TTA}-cRGDfK group (FIG. 20C). These data suggest that the combination of caging and local phototriggering of NP_{TTA}-c[R]GDfK decreased off-target binding in liver and spleen and provided a higher specificity to tumor, compared to only using targeted nanoparticles (NP_{TTA}-cRGDfK).

FIG. 20 shows the effect of phototriggering on the biodistribution of NP_{TTA}-c[R]GDfK. FIG. 20A shows representative fluorescence images of organs and tumors 24 h after intravenous injection. FIG. 20B shows biodistribution of injected formulations in animals with U87 glioblastomas. Results are the mass of DiR per gram of various tissues. FIG. 20C shows the ratios of the DiR concentrations in liver and spleen to that in tumor, compared in particles with uncaged ligands (NP_{TTA}-cRGDfK) and particles with caged ligands (NP_{TTA}-c[R]GDfK) after irradiation at the tumor site (+LED). NP_{TTA}: particles without targeting ligands; LED: irradiation with 530 nm light, 200 mW/cm², 5 min. Data are means+/−SD, n=4. **p<0.005, *p<0.05.

Histological studies of irradiated skin above the tumor were conducted to assess the phototoxicity of the 530 nm light. Mice were euthanized 24 h after injection, and the skins were harvested for hematoxylin & eosin (H&E) staining. Mice in the NP_{TTA}-c[R]GDfK and NP_{TTA}-c[R]GDfK+LED groups did not show any histological changes in the skins, indicating no significant phototoxicity caused by the 530 nm irradiation.

### Example 13

For light-triggered drug delivery systems, the wavelength used for photoresponsiveness is of great importance. To date, the most frequently used is UV light (<400 nm), which has a quite limited depth of tissue penetration, and can cause severe phototoxicity. Light in the NIR window (650 nm-900 nm) would allow deeper tissue penetration than UV light, but the photon energy is too low to break covalent bonds directly. Upconversion using rare-earth doped particles has been exploited to convert the low-energy NIR light to high-energy UV light. However, the upconversion quantum efficiency is quite low, so that high irradiances and long periods of irradiation have been required which can generate burns. In addition to the limitations of tissue penetration and light-induced tissue injury, there is the clinically important parameter of irradiation time: shorter is better for patient comfort, convenience, and in some cases for procedural feasibility. Achieving effective irradiation in short times can be challenging, since the amount of light delivered is obviously dependent on the duration of irradiation.

The above examples demonstrated in vivo tumor phototargeting of nanoparticles enabled by TTA-UC in a subcutaneous tumor model. The nanoparticles employed in some of these examples could be triggered effectively at low irradiance (200 mW/cm²) over a short time frame (5 min), without tissue injury. Moreover, the triggering could be achieved with a LED, which is inexpensive and easily portable. Phototargeting enhanced specific accumulation of nanoparticles in the tumor but not in the liver or spleen, that is, accumulation in off-target tissues was minimized.

The green light used here to trigger nanoparticles has limited tissue penetration, and may be most relevant for drug delivery to superficial or easily accessible tissues, such as the skin or the retina. The wavelengths at which systems like the one described here could operate would depend on the selected sensitizer and annihilator pairs and the specific photocleavable groups.

### Example 14

Following are additional materials and methods used in the above examples.

Materials. Chemicals were purchased from Sigma-Aldrich (St. Louis, Mo.) and used without further purification unless otherwise stated. Poly(D,L-lactic acid)(2000)-poly(ethylene oxide)(3000)-N-hydroxysuccinimide (PLA-PEG-NHS) and PLA(2000)-methoxy PEG (mPEG, 2000) (PLA-mPEG) were purchased from Advanced Polymer Materials (Dorval, Montreal, Canada). 7-diethylamino-4-hydroxymethylcoumarin was purchased from INDOFINE Chemical Company (Hillsborough, N.J.). Lissamine™ rhodamine B ethylenediamine (LRB), 1,1′-dioctadecyl-3,3,3′,3′-tetramethylindotricarbocyanine iodide (DiR) and cyclo-(RGDfK) were purchased from AnaSpec (Fremont, Calif.). c[R]GDfK was acquired from AnaSpec by custom synthesis. Human glioblastoma (U87) cells were purchased from American Type Culture Collection (ATCC) (Manassas, Va.). The CellTiter 96® AQueous One Solution Cell Proliferation Assay solution was from Promega (Medison, Wisconsin). Other cell culture agents were purchased from Thermo Fisher Scientific (Waltham, Mass.).

Preparation of polymeric micelles. Polymers of PLA-PEG-c[R]GDfK, PLA-PEG-cRGDfK and PLA-PEG-LRB were synthesized as discussed above. To prepare NP_{TTA}-c[R]GDfK, PLA-PEG-c[R]GDfK (2.0 mg), PLA-mPEG (8.0 mg) and DiR (0.1 mg) were co-dissolved in 5 mL of acetonitrile, then mixed with 0.5 mL of DPA (2.0 mM), 0.05 mL of PdOEP (0.2 mM) toluene solutions. Rotary evaporation was used to slowly remove the solvents at 45° C. The dried polymer film was hydrated with 2 mL of PBS at 60° C. For other TTA-containing micelles, the same procedure was used except the use of different composition of polymers for each micelles, NP_{TTA}: PLA-mPEG (10.0 mg); NP_{TTA}-cRGDfK: PLA-PEG-cRGDfK (2.0 mg) and PLA-mPEG (8.0 mg); LRB-labelled NP_{TTA}-c[R]GDfK (no DiR): PLA-PEG-c[R]GDfK (2.0 mg), PLA-PEG-LRB (1.0 mg) and PLA-mPEG (7.0 mg).

Spectroscopic characterization. The fluorescence lifetime was measured on an Edinburgh FL-920 fluorescence spectrometer (Livingston, United Kingdom) with a semiconductor laser as the excitation source (excitation wavelength at 379 nm). The fluorescent spectra and UV-Vis spectra were recorded by an Agilent Cary Eclipse fluorescence spectrophotometer (Santa Clara, Calif.) and an Agilent 8453 UV-Vis G1103A spectrophotometer (Santa Clara, Calif.).

Lifetime analysis. A bi-exponential fit was employed to analyze the lifetime of DPA fluorescence. Both the fast and the slow components were presented in Table 2, as well as the fractional contributions of the fast and slow components. The lifetime was calculated according to the following equation. The photon-weighted average lifetime is as follows:

$$τ = \frac{{τ}_{fast}^{2} \times {P}_{fast} + {τ}_{slow}^{2} \times {P}_{slow}}{{τ}_{fast} \times {P}_{fast} + {τ}_{slow} \times {P}_{slow}}$$

TABLE 2

| | | | τ_{slow} | | | |
|---|---|---|---|---|---|---|
| Sample | τ_{fast} (ns) | P_{fast} | (ns) | P_{slow} | χ² | τ (ns) |
| c[R]GDfK-NP_{TTA} | 1.41 | 42% | 4.35 | 58% | 0.91 | 3.80 |
| cRGDfK-NP_{TTA} | 2.35 | 49% | 7.42 | 51% | 0.98 | 6.20 |

λₑₓ = 375 nm,
λₑₘ = 410 nm.

τ_{fast} and P_{fast} represent the lifetime and the fractional contribution of the fast component of the decay, respectively. τ_{slow} and P_{slow} represent the lifetime and the fractional contribution of the slow component of the decay, respectively. T is the photon-weighted average lifetime of DPA calculated as described by the above equation. χ² is the fit quality criterion.

The FRET efficiency (E) was determined according to the following equation:

E=1−τ_{DA}/τ_{D},

where E indicates the percentage of excitation photons that contribute to FRET, was 39%.

Photocleavage of DEACM from NPTTA-c[R]GDfK. To evaluate the photorelease rate of DEACM-OH from NP_{TTA}-c[R]GDfK, 1 mL of NP_{TTA}-c[R]GDfK solution (1 mg mL⁻¹) was placed in a quartz cuvette and irradiated with LED light (530 nm, 200 mW cm⁻²). At each irradiation time, the solution was transferred into an Amicon® Ultra centrifugal filter (MWCO: 50,000 Da) and centrifuged at 4000 rmp for 15 min. The fluorescence of the filtrate was measured by a Cary Eclipse fluorescence spectrophotometer (Agilent, CA, USA).

Cell viability assay. U87 cell viability was determined by MTS. 5000 cells were seeded into 96 well microplates (Costar, Corning, N.Y.) and grown in DMEM medium containing 10% (v/v) fetal bovine serum (FBS) and 1% penicillin Streptomycin (Invitrogen) for 24 h. To assess the cytotoxicity of the light itself, cells were irradiated with 385 nm or 530 nm LED at different irradiances for 5 min. After 24 h of incubation, cell viability was assessed.

To assess the cytotoxicity of nanoparticles and free DEACM-OH, the culture medium was replaced with the medium containing 0.25, 0.50, and 1.0 mg/mL nanoparticles (NP_{TTA}, NP_{TTA}-cRGDfK and NP_{TTA}-c[R]GDfK) or free DEACM-OH (1.0, 10, 50, 100 micromolar). Cell viability was assessed after 24 or 72 h of incubation. In the NP_{TTA}-c[R]GDfK+LED group, cells were irradiated for 5 min with a 530 nm LED (200 mW/cm²) after application of medium containing NP_{TTA}-c[R]GDfK. At 24 h or 72 h the cell viability was assessed.

Phototargeting in vitro. U87 cells were incubated with media containing NP_{TTA}-c[R]GDfK (1.0 mg/mL in phosphate buffer saline) labeled with the fluorescent probe Lissamine Rhodamine B (LRB). The culture dish (8.5 cm) was covered with an aluminum foil mask only allowing light penetration in a 3 mm diameter area. The dish was placed under a fluorescence microscope and left unperturbed for 10 min to avoid medium migration prior to light irradiation (530 nm, 200 mW/cm², 5 min). After irradiation, the dish was left unperturbed for another 25 min and unbound nanoparticles were carefully removed.³

Phototargeting in vivo. The study protocol was reviewed and approved by the Institutional Animal Care and Use Committee of Boston Children's Hospital. Six to eight-week-old nu/nu nude mice were purchased from Charles River Laboratories and maintained under pathogen-free conditions for the animal study. For subcutaneous U87 tumor models, 5×10⁶ cells/0.1 mL U87 cells in medium were injected subcutaneously at the right shoulder. Tumor dimensions were measured with calipers and the volume was determined by the following:

Volume=length*width*width/2.

When the tumor volume reached ˜100-200 mm³, the mice were administered intravenously (i.v.) under isoflurane anesthesia 5.0 mg/mL NP_{TTA}, NP_{TTA}-cRGDfK or NP_{TTA}-c[R]GDfK (200 microliters). In photo-triggered targeting experiments, the tumor site was irradiated with 530 nm light for 5 min (200 mW/cm²) at 1 min after the injection. Fluorescent imaging was carried out by an IVIS imaging system (IVIS spectrum, Caliper Life Sciences) with the ICG channel.

Biodistribution study. Mice were euthanized 24 h post-injection and organs were collected. Tissues were weighed and sonicated in 500 microliters 5% triton solution (Sigma Aldrich) in ice for 2 min, then the same volume of methanol was added to extract the DiR and another 2 min sonication was performed. Mixtures were vortexed for 2 min and then centrifuged at 14,000 rpm for 15 min (Microfuge 22R Centrifuge, Beckman Coulter, Brea, Calif.). To determine the content of DiR in each tissue homogenate sample, 400 microliters of the supernatant solution was transferred into a cuvette and analyzed by a fluorescence spectrometer (Agilent, California). The data were divided by tissue mass (micrograms/g).

Histological study. Mice were euthanized 24 h after injection, and the skin above tumors and tumors were harvested. The skin tissues were formalin-fixed and paraffin-embeded. Tissue blocks were sectioned, stained with hematoxylin & eosin (H&E) stain and studied by light microscopy.

Statistics. All data were reported as means+/−standard deviations (SD). p values were calculated by unpaired t-tests using Origin 8.0 software (Massachusetts, USA). Statistical significance was determined when p<0.05.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A composition, comprising:
a photosensitizer;
an annihilator able to accept triplet-triplet energy transfer from the photosensitizer;
a cleavable moiety able to accept energy from the annihilator in the higher energy state to cause cleavage of the cleavable moiety; and
a releasable moiety releasable from the composition upon cleavage of the cleavable moiety.

2. (canceled)

3. The composition of claim 1, wherein the photosensitizer is able to absorb a photon and transfer energy from the photon to the annihilator.

4. The composition of claim 1, wherein the photosensitizer comprises palladium octaethylporphyrin.

5-8. (canceled)

9. The composition of claim 1, wherein the photosensitizer has an excitation wavelength of between about 360 nm and about 700 nm.

10-11. (canceled)

12. The composition of claim 1, wherein the photosensitizer has an excitation wavelength greater than the emission wavelength of the annihilator.

13. The composition of claim 1, wherein the photosensitizer is a transition metal-porphyrin.

14-15. (canceled)

16. The composition of claim 1, wherein the annihilator is able to accept triplet-triplet energy transfer from the photosensitizer after absorption of the photon by the photosensitizer to produce a higher energy state.

17. (canceled)

18. The composition of claim 1, wherein the annihilator is able to transfer energy to the cleavable moiety.

19. (canceled)

20. The composition of claim 1, wherein two annihilator molecules, each at a triplet energy state, participate in triplet-triplet annihilation to produce a first annihilator molecule having higher energy that can be transferred to the cleavable moiety, and a second annihilator having a lower energy state.

21. The composition of claim 1, wherein the annihilator comprises 9,10-diphenylanthracene.

22-26. (canceled)

27. The composition of claim 1, wherein the annihilator has an emission wavelength of between about 360 nm and about 700 nm.

28. The composition of claim 1, wherein the cleavable moiety has absorption overlapping with the upconversion emission from the annihilator.

29-30. (canceled)

31. The composition of claim 1, wherein the cleavable moiety comprises an arylcarbonylmethyl moiety.

32-52. (canceled)

53. The composition of claim 1, wherein the releasable moiety is a caged species.

54-71. (canceled)

72. A method, comprising:
absorbing a photon in a photosensitizer;
transferring energy from the photosensitizer to an annihilator via triplet-triplet energy transfer;
producing a higher-energy state via triplet-triplet annihilation from the transferred energy in two annihilators;
transferring energy from the annihilator in the higher-energy state to an active moiety via Förster resonance energy transfer; and
causing a chemical reaction in the active moiety using the transferred energy.

73. The method of claim 72, wherein the active moiety is a cleavable moiety, and the chemical reaction is cleavage of the cleavable moiety.

74. The method of claim 72, wherein cleaving the cleavable moiety causes release of a releasable moiety.

75. A method, comprising:
applying, to an eye of a subject, a composition comprising a photosensitizer, an annihilator able to accept triplet-triplet energy transfer from the photosensitizer, and a cleavable moiety able to accept energy from the annihilator in the higher energy state to cause cleavage of the cleavable moiety; and
applying light to at least a portion of the eye to cause cleavage of the cleavable moiety.

76. The method of claim 75, wherein the light is coherent.

77. (canceled)

78. The method of claim 75, wherein the light is applied to the eye at an irradiance of at least about 1 mW/cm².

79-84. (canceled)
